(19) United States
(12) Patent Application Publication
(10) Pub. No.: US 2015/0273025 A1
(43) Pub. Date: Oct. 1, 2015

(54) APYRASE TREATMENTS

(71) Applicant: THE REGENTS OF THE UNIVERSITY OF MICHIGAN, Ann Arbor, MI (US)

(72) Inventors: Chuanwu Xi, Ann Arbor, MI (US); Benjamin Levi, Ann Arbor, MI (US); Jianfeng Wu, Ann Arbor, MI (US); Stewart C. Wang, Ann Arbor, MI (US)

(21) Appl. No.: 14/431,171

(22) PCT Filed: Sep. 27, 2013
(86) PCT No.: PCT/US2013/062337

§ 371 Date: Mar. 25, 2015

Related U.S. Application Data
(60) Provisional application No. 61/707,228, filed on Sep. 28, 2012.

(51) Int. Cl. A61K 38/46; A61K 31/795; A61K 45/06; A61K 38/12

(52) U.S. Cl. CPC A61K 38/46; A61K 38/12; A61K 31/795; A61K 45/06; C12Y 306/01005

(57) ABSTRACT

Provided herein is technology relating to compositions comprising apyrase and particularly, but not exclusively, to compositions comprising apyrase, methods of treating with apyrase, and uses of apyrase related to treating a subject, e.g., a subject suffering from a microbial infection, a subject who has a wound or burn and is in need of an antimicrobial treatment, and/or a subject in need of treatment for heterotopic ossification.

This application claims priority to U.S. provisional patent application 61/707,228, filed Sep. 28, 2012, which is incorporated herein by reference in its entirety.

### STATEMENT REGARDING FEDERALLY SPONSORED RESEARCH OR DEVELOPMENT

This invention was made with government support under GM098350 awarded by the National Institutes of Health. The government has certain rights in the invention.

### FIELD OF TECHNOLOGY

Provided herein is technology relating to compositions comprising apyrase and particularly, but not exclusively, to compositions comprising apyrase, methods of treating with apyrase, and uses of apyrase related to treating a subject, e.g., a subject suffering from a microbial infection, a subject who has a wound or burn and is in need of an antimicrobial treatment, and/or a subject in need of treatment for heterotopic ossification.

### BACKGROUND

The term “antibiotic” is broadly defined as a chemical compound produced by one microorganism that inhibits the growth of a different microorganism. Today, there are more than 150 antibiotics classified by their chemical structures and mechanisms of action.

When the first therapeutic antibiotic, penicillin, was introduced in the early 1940s, many believed that the threat from infectious diseases was over. However, in the past 25 years, through the abuse and misuse of antibiotics, many bacteria have developed resistance to these antibiotics. Studies over the past decades have documented the extensive human and economic toll of antibiotic resistance. When the problem was first widely recognized as an emergent medical issue, antibiotic-resistant microorganisms added an estimated $200 million per year to medical bills. When costs for extended hospital stays are considered, the estimated costs increased by $30 billion per year (Phelps, Medical Care, 27:194-203 (1989)). More recent studies show that methicillin-resistant Staphylococcus aureus (MRSA) alone caused 250,000-300,000 hospital acquired infections, about 2.7 million hospital days, and 12,000 deaths annually, resulting in annual costs of about $9.5 billion in the early 2000s and that vancomycin-resistant enterococci (VRE) caused about 26,000 infections in US hospitals in 2004.

Resistance to antimicrobial agents (e.g., antibiotic resistance) refers to a type of drug resistance where a microorganism is able to survive exposure to an antimicrobial agent such as an antibiotic intended to inhibit the growth of the microorganism or to kill it. Antibiotic resistance may arise by spontaneous or induced genetic mutation in a microorganism. Extensive research focused on the mechanisms of resistance has developed several physiological models for resistance:
(1) Loss of cell permeability to the antibiotic;
(2) Enzymes that render the antibiotic ineffective;
(3) Export of the antibiotic out of the cell once it enters the cell;
(4) Modification of the target of the antibiotic; and
(5) Modification of metabolic pathways which result in by-passing the reaction inhibited by the antibiotic.
   Genes conferring resistance to antimicrobial agents by these mechanisms, like the antibiotics themselves, are extant in populations of microorganisms.

The seriousness of these mechanisms of antibiotic resistance is accentuated by the ability of the bacteria to transfer the resistance to other microorganisms, some of which may be fairly genetically unrelated to the antibiotic-resistant strain. In particular, a gene for antibiotic resistance that evolves via natural selection may be shared. Evolutionary stress such as exposure to antibiotics then selects for the antibiotic resistant trait. Many antibiotic resistance genes reside on plasmids, facilitating their transfer. If a bacterium carries several resistance genes, it is called multidrug resistant (MDR). The antibiotic-resistance transfer can occur through conjugation, transduction, transformation, or transposition.

As a consequence, today there are strains of virtually every major bacterial human pathogen that are resistant to some of the most effective antibiotics. Examples of medically relevant bacteria being detected in clinics with increasing resistances to antibiotics include Staphylococcus aureus, Streptococcus spp., Enterococcus spp., Pseudomonas aeruginosa, Clostridium difficile, Salmonella spp., Escherichia coli, Acinetobacter baumannii, and Mycobacterium tuberculosis. These pathogens can cause diarrhea, urinary tract infections, otitis media, meningitis, tuberculosis, gonorrhea, pneumonia, dysentery, wound infections, septicemia, bacteremia, and surgical infections (see, e.g., Lippe, “Breakout: The Evolving Threat of Drug-Resistant Diseases”, Sierra Clubs, San Francisco (1995)).

Thus, while 90% of bacterial infections are successfully treated with first line antibiotics, there are increasingly situations in which over 40% of the infections are resistant to one or more antibiotics (including second-line products). Some of the newest antibiotics, e.g., vancomycin, have been shown to be the only antibiotics that are effective against some pathogenic bacteria. It has become the last line of defense against some infections, particularly those by methicillin-resistant Staphylococcus aureus.

However, resistance to these last lines of defense is now also becoming dangerously more prevalent. For example, some less pathogenic strains of the genus Enterococcus have vancomycin-resistance genes, and have been shown, in the laboratory, to transfer this resistance to Staphylococcus strains (see, e.g., Noble W C, et al. (1992) “Co-transfer of vancomycin and other resistance genes from Enterococcus faecalis NCTC 12201 to Staphylococcus aureus” FEMS Microbiol Lett. 72: 195-8). Subsequently, vancomycin-resistant strains of Staphylococcus have been isolated from clinical specimens (Chang S, et al. (2003) “Infection with vancomycin-resistant Staphylococcus aureus containing the vanA resistance gene”. N Engl. J. Med. 348: 1342-7). As a result, as resistances to our antibiotics of last resort spread throughout populations of pathogens, future physicians will have no treatment for infections by dangerous strains of microbes.

Resistance is likely resulting from widespread antibiotic use in human medicine, veterinary medicine, agriculture, and livestock production. Any use of antibiotics can increase selective pressure in a population of bacteria to allow the resistant bacteria to thrive and the susceptible bacteria to die off. As resistance towards antibiotics becomes more common, a greater need for alternative treatments arises. However, despite a push for new antibiotic therapies there has been a continued decline in the number of newly approved drugs. Thus, antibiotic resistance poses a significant problem. There is a crucial need for novel antimicrobial agents that effectively inhibit the growth of infectious and pathogenic microbes as well as for novel strategies for improving the effectiveness of extant antimicrobial agents.

### SUMMARY

Provided herein is technology relating to compositions comprising apyrase and particularly, but not exclusively, to compositions comprising apyrase, methods of treating with apyrase, and uses of apyrase related to treating a subject, e.g., a subject suffering from a microbial infection, a subject who has a wound or burn and is in need of an antimicrobial treatment, and/or a subject in need of treatment for heterotopic ossification.

Wound infections are commonplace and represent a major cause of morbidity and mortality. Open injuries have a potential for serious bacterial wound infections, including gas gangrene and tetanus, and these in turn may lead to long term disabilities, chronic wound or bone infection, and death. In the United States, bacterial wound infections affect millions of people every year and incur considerable economic loss. In surgical site infections alone, costs per patient increased by an average of $20,842 as a result of infection (see, e.g., Sen et al. (2009) “Human skin wounds: a major and snowballing threat to public health and the economy”, Wound Repair Regen, 7(6): 763-71). In aggregate, it has been estimated that an excess of US $25 billion is spent on the treatment of chronic wounds alone while healthcare costs and an aging population are rapidly on the rise (see, e.g., Sen et al. (2009) “Human skin wounds: a major and snowballing threat to public health and the economy”, Wound Repair Regen, 7(6): 763-71). Wound infections also affect military operations, homeland preparedness against natural disasters, and preparedness against terrorism attacks. In both general public and wartime or natural disaster emergency wound care, effective firsthand measures are required to prevent and treat wound infections—failure of which could result in the development of chronic wound infections that are difficult to eradicate. This necessity is especially pronounced given the current emergence of multi-drug resistant bacteria. In some embodiments, the technology provided herein addresses these issues by developing a drug candidate that enhances the prevention of bacterial wound infections and also the efficacy of antimicrobial agents. In particular, some embodiments of the technology provide apyrase (e.g., a human derived apyrase) for drugs targeting wound infections.

Recently, the emergence of multidrug resistant (MDR) bacterial strains has become a challenge for antimicrobial chemotherapy of patients, including soldiers with infected extremity wounds returning from Iraq and Afghanistan (Calhoun et al “Multidrug-resistant organisms in military wounds from Iraq and Afghanistan” Clin Orthop Relat Res 466(6): 1356-62). Frequently identified MDR wound infection pathogens include A. baumannii, S. aureus (MRSA, VRSA), P. aeruginosa, K pneumoniae, and extended-spectrum beta-lactamase (ESBL)-producing E. coli, collectively called “ESKAPE” pathogens. These bacteria are found in both acute and chronic wounds and are a formidable threat to wound healing. Certain strains of A. baumannii, P. aeruginosa, and K. pneumonia have become resistant to colistin, an antibiotic considered a last line of defense against many Gram-negative MDR infections (Lim et al (2010) “Resurgence of colistin: a review of resistance, toxicity, pharmacodynamics, and dosing” Pharmacotherapy 30(12): 1279-91). Therefore, it is critically important to develop new measures to eradicate infections by these pathogens and reduce reoccurrences.

The use of apyrase as a drug is based on the results of experiments performed during the development of embodiments of the technology provided herein. In particular, apyrase re-sensitizes bacteria (both drug sensitive and resistant strains) to antibiotics such as colistin. Consequently, drug products containing apyrase fight a major issue in infectious diseases: multidrug resistance.

Accordingly, some embodiments of the technology provide a human derived apyrase as a drug substance to target eATP for control of bacterial infections. Apyrase increases the effectiveness of currently used antibiotics and gives physicians another tool to prevent the development of infection. In some embodiments, apyrase delivered in conjunction with antibiotics enhances eradication of antibiotic-resistant bacteria. Combinatorial use of apyrase and with other antibiotic treatments improves the treatment of a range of infections, including flesh wounds and multi-site or even bloodstream bacterial infections, by medical personnel. It is contemplated that embodiments of the technology are used to treat nosocomial pneumonia, bacteremia and sepsis, infections in cystic fibrosis, catheter-associated bacteriuria, etc., especially when the last line of antibiotics is no longer effective. Patients and providers in the healthcare industry benefit from the integration of apyrase into current treatment protocols for wounds and potentially more serious bacterial infections. Insurance companies benefit from reduced infection costs, which may help lower the overall health care costs of the public. Pharmaceutical companies and/or others engaged in research, development, and product marketing also benefit by delivering cost-saving drugs with enhanced efficacy.

Some embodiments of the technology relate to a composition comprising apyrase and an antimicrobial agent. In some embodiments, the antimicrobial agent is colistin and in some embodiments the antimicrobial agent is an antibacterial polymer, e.g., polymer E2 or polymer E4 as described herein. The technology is not limited in the concentrations of apyrase and/or antimicrobial agent that are used. These concentrations may vary by application and use and may, in some embodiments, be determined by one of skill in the art based on various tests and/or criteria, e.g., sex, weight, severity of the infection or ossification, age, other health conditions present, etc. For example, in some embodiments, the apyrase has a concentration of from 0.01 U/ml to 50 U/ml, more particularly from 0.01 U/ml to 10 U/ml, and in some embodiments the apyrase has a concentration from approximately 1 to approximately 2 U/ml.

The technology is not limited in the antimicrobial agent that is used, and, indeed, the technology encompasses any antimicrobial agent that is known or unknown, extant or yet to be discovered. By way of example, embodiments of the technology comprise an antimicrobial agent from a class that is a macrolide, penicillin, cephalosporin, carbepenem, monobactam, beta-lactam inhibitor, oxaline, aminoglycoside, chloramphenicol, sulfonamide, glycopeptide, quinolone, tetracycline, fusidic acid, trimethoprim, metronidazole, clindamycin, mupirocin, rifamycins, streptogramin, lipoprotein, polyene, azole, or echinocandin. Furthermore, some embodiments of the technology comprise an antimicrobial agent that is erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, or nystatin.

The technology encompasses medical uses in which apyrase and an antimicrobial agent are incorporated, attached, or otherwise associated with a device, material, surface, substance, etc. For example, some embodiments relate to a material that is a dressing, a gel, an ointment, a bandage, a solution, a cream, a salve, or a spray comprising apyrase and an antimicrobial compound.

In another aspect, the technology relates to a method for treating or preventing an infection comprising identifying a subject in need of an antimicrobial treatment and administering to the subject an apyrase and an antimicrobial agent. In some embodiments the administering comprises administering a composition comprising the apyrase and the antimicrobial agent and in some embodiments the administering comprises administering a first composition comprising the apyrase and administering a second composition comprising the antimicrobial agent. Furthermore, embodiments are provided wherein the first composition and the second composition are administered sequentially and embodiments are provided wherein the first composition and the second composition are administered simultaneously. The embodiments of the technology related to methods of treating or preventing an infection are not limited in the antimicrobial agents that are administered. For example, in some embodiments, the antimicrobial agent is colistin, antimicrobial polymer E2, or antimicrobial polymer E4. Further examples of antimicrobial agents are compounds classified as a macrolide, penicillin, cephalosporin, carbepenem, monobactam, beta-lactam inhibitor, oxaline, aminoglycoside, chloramphenicol, sulfonamide, glycopeptide, quinolone, tetracycline, fusidic acid, trimethoprim, metronidazole, clindamycin, mupirocin, rifamycins, streptogramin, lipoprotein, polyene, azole, or echinocandin. Additional specific antimicrobial agents that find use within the scope of the technology are erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, and nystatin.

Some embodiments provide methods further comprising testing the subject for an infection. In some embodiments, the testing is before the administering and in some embodiments the testing is after the administering. In some embodiments, a second treatment (e.g., of an apyrase and an antimicrobial agent) is administered to the subject. For example, some embodiments provide that a dosage of the second administering is determined based on a result of a testing. Embodiments are provided in which the administering occurs by a route selected from the group consisting of oral, intravenous, intraperitoneal, intramuscular, transdermal, subcutaneous, topical, sublingual, and rectal. In addition, embodiments are provided wherein the administering comprises contacting a subject with a material selected from the group consisting of a dressing, a gel, an ointment, a bandage, a solution, a cream, a salve, and a spray, wherein the composition is a component of the material.

The methods are not limited in the types of subjects and patients that are appropriate for the described administrations. In some embodiments, the subject is in need of a treatment for a microbial infection and in some embodiments the subject is in need of a treatment to prevent a microbial infection. For example, in some embodiments the subject has a wound and in some embodiments the subject has a burn. In some embodiments the subject is in need of a treatment for heterotopic ossification. As such, embodiments are provided for methods comprising identifying a subject in need of a treatment for heterotopic ossification; and administering to the subject an agent that hydrolyzes a compound selected from the group consisting of ATP, dATP, an analog of ATP, and a derivative of ATP, e.g., an apyrase. Embodiments of the methods relate to decreasing an activity of a mesenchymal stem cell. In some embodiments, the activity of the mesenchymal stem cell is osteogenesis. In some embodiments, the method comprises inhibiting a bone morphogenic protein signaling pathway. In related embodiments, the subject who is treated has experienced a trauma, burn, upper extremity injury, or surgery. In some embodiments, the subject has a soft-tissue trauma, an amputation, a central nervous system injury, a vasculopathy, an arthroplasty, or an end-stage cardiac valve disease.

Embodiments of the technology also relate to compositions comprising apyrase, wherein the composition has antimicrobial activity and/or has anti-osteogenic activity. Accordingly, embodiments are provided for a method of treating a wound or a burn, the method comprising selecting a subject who is in need of an antimicrobial and an anti-osteogenic treatment and administering to the subject a composition comprising apyrase. In some embodiments, the subject has a wound or a burn or has had a surgery. The technology provided herein relates to preventive medicine and a treatment drug for wound infections. Accordingly, in some embodiments the technology encompasses cloning and/or engineering a human-derived apyrase in an expression system (e.g., a yeast expression system) and producing apyrase in a mammalian cell line. As such, in some embodiments are provided compositions comprising a human apyrase, an engineered human apyrase, a eucaryotic (e.g., mammalian) apyrase, and the like.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a plot showing that apyrase improved the efficiency of killing A. baumannii strain ATCC 19606 by colistin.

FIG. 2 is a plot showing that apyrase improved the efficiency of killing A. baumannii strain DMC1 by colistin.

FIG. 3 is a plot showing that apyrase improved the efficiency of killing P. aeruginosa strain ATCC 27853 by colistin.

FIG. 4 is a plot showing that apyrase improved the efficiency of killing E. coli strain K12 by colistin.

FIG. 5 is a plot showing that apyrase improved the efficiency of killing P. aeruginosa strain PAO300 by colistin.

FIG. 6 is a plot showing that apyrase improved the efficiency of killing A. baumannii strain VBA9 by colistin.

FIG. 7 is a plot showing that apyrase improved the efficiency of killing A. baumannii strain AC285 by colistin.

FIG. 8 is a plot showing that that apyrase improved the efficiency of killing A. baumannii strain AC121 by colistin.

FIG. 9 is a plot showing that apyrase improved the efficiency of killing P. aeruginosa strain AU1292 by colistin.

FIG. 10 is a plot showing that apyrase improved the efficiency of killing P. aeruginosa strain AU7443, mucoid by colistin.

FIG. 11 is a plot showing that apyrase improved the efficiency of killing Burkholderia vietnamiensis AU10214 by colistin.

FIG. 12 is a plot showing that apyrase improved the efficiency of killing Burkholderia cepacia AU1114 by colistin.

FIG. 13 is a plot showing that apyrase improved the efficiency of killing P. aeruginosa strain AU8104 by colistin.

FIG. 14 is a plot showing that apyrase improved the efficiency of killing Burkholderia cepacia GIIIa AU0019 by colistin.

FIG. 15 is a plot showing that apyrase improved the efficiency of killing Burkholderia cepacia GIIIb AU0062 by colistin.

FIG. 16 is a plot showing that apyrase improved the efficiency of killing Burkholderia ambifaria AU5203 by colistin.

FIG. 17 is a plot showing that apyrase improved the efficiency of killing Burkholderia cepacia GIIIb AU0055 by colistin.

FIG. 18 is a plot showing that apyrase improved the efficiency of killing P. aeruginosa AU0584 by colistin.

FIG. 19 is a plot showing that apyrase improved the efficiency of killing Burkholderia dolosa AU0589 by colistin.

FIG. 20 is a plot showing that apyrase improved the efficiency of killing Burkholderia multivorans AU0801 by colistin.

FIG. 21 is a plot showing that apyrase improved the efficiency of killing E. coli K12 by antimicrobial polymer E2.

FIG. 22 is a plot showing that apyrase improved the efficiency of killing S. aureus ATCC 25923 by antimicrobial polymer E2.

FIG. 23 is a plot showing that apyrase improved the efficiency of killing Mycobacterium immunogenum ATCC 700505 by antimicrobial polymer E4.

FIG. 24 is a plot showing that apyrase does not affect bacterial growth.

FIG. 25 is a plot showing increased permeabilization of the bacterial membrane in the presence of colistin and apyrase.

FIG. 26 is a plot showing that apyrase and colistin effectively kill A. baumannii ATCC 17978 on skin using an in vivo burn model.

FIG. 27 is a plot showing that apyrase and colistin effectively kill A. baumannii ATCC DMC1 on skin using an in vivo burn model.

FIG. 28 is a plot showing that apyrase and colistin effectively kill A. baumannii ATCC DMC1 on skin using an in vivo burn model.

FIG. 29 is a plot showing an increase in osteogenic differentiation of MSCs after burn injury and an amelioration of the increase by apyrase.

FIG. 30 is a plot showing that mice with a burn injury develop significantly more bone and develop bone at an earlier time point than non-burn control mice.

FIG. 31 is a series of plots showing dose-kill curves for four different strains when exposed to colistin alone or to the combination of colistin and apyrase.

FIG. 32 is a plot showing the viable bacterial number 24 hours after treatment of a burn with apyrase and apyrase plus colistin.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to compositions comprising apyrase and particularly, but not exclusively, to compositions comprising apyrase, methods of treating with apyrase, and uses of apyrase related to treating a subject, e.g., a subject suffering from a microbial infection and/or a subject who has a wound or burn and is in need of an antimicrobial treatment. In some embodiments, the technology relates to compositions, methods, and uses related to a subject suffering from heterotopic ossification.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The terms “bacteria” and “bacterium” refer to prokaryotic organisms of the domain Bacteria in the three-domain system (see Woese C R, et al., Proc Natl Acad Sci USA 1990, 87: 4576-79). It is intended that the terms encompass all microorganisms considered to be bacteria including Mycobacterium, Mycoplasma, Chlamydia, Actinomyces, Streptomyces, and Rickettsia. All forms of bacteria are included within this definition including cocci, bacilli, spirochetes, spheroplasts, protoplasts, etc. In some embodiments, bacteria are capable of causing disease and product degradation or spoilage.

As used herein, a “pathogen” is an organism or agent that is capable of causing a disease. The terms “non-pathogenic microbe” or “non-pathogenic microorganism” include all known and unknown non-pathogenic microbes (Bacteria, Archaea, and/or Eukarya) and any pathogenic microbe that has been mutated or converted to a non-pathogenic state. Furthermore, a skilled artisan recognizes that some microbes may be pathogenic to specific species and non-pathogenic to other species; thus, these microbes can be utilized in the species in which it is non-pathogenic or mutated so that it is non-pathogenic.

“Strain” as used herein in reference to a microorganism describes an isolate of a microorganism (e.g., bacteria, virus, fungus, parasite) considered to be of the same species but with a unique genome and, if nucleotide changes are non-synonymous, a unique proteome differing from other strains of the same organism. Strains may differ in their non-chromosomal genetic complement. Typically, strains are the result of isolation from a different host or at a different location and time, but multiple strains of the same organism may be isolated from the same host.

As used herein, the term “infection” refers to the invasion of a host animal by pathogenic microorganisms such as bacteria. For example, the infection may include the excessive growth of microorganisms that are normally present in or on the body of an animal or growth of microorganisms that are not normally present in or on the animal. More generally, an infection can be any situation in which the presence of a microorganism population(s) is damaging to a host animal. Thus, an animal is “suffering” from an infection when an excessive amount of a bacterial population is present in or on the animal's body, or when the presence of a microorganism population(s) is damaging the cells or other tissue of the animal.

As used herein, the term “persistent infection” refers to an infection that is not completely eradicated through standard treatment regimens using anti-microbial agents. Persistent infections are caused by microorganisms capable of establishing a cryptic or latent phase of infection and may be classified as such by culturing the microorganism from a patient and demonstrating survival in vitro in the presence of antimicrobial agents or by determination of antimicrobial treatment failure in a patient. An in vivo persistent infection can be identified through the use of a reverse transcriptase polymerase chain reaction (RT-PCR) to demonstrate the presence of 16S rRNA transcripts in infected cells after treatment with anti-microbial agents (see, e.g., Antimicrob. Agents Chemother. 12: 3288-97, 2000).

As used herein, the term “apyrase” refers to any enzyme that hydrolyzes ATP to release AMP and phosphate, without regard to the origin or type of the enzyme (e.g., purified, recombinant, existing in nature, engineered, truncated, mutated).

As used herein, the term “subject” refers to individuals (e.g., human, animal, or another organism) to be treated by the methods or compositions of the present technology. Subjects include, but are not limited to, mammals (e.g., murines, simians, equines, bovines, porcines, canines, felines, and the like), and most preferably includes humans. In the context of the technology, the term “subject” generally refers to an individual who will receive or who has received treatment for a condition characterized by the presence of an infectious microbe, or in anticipation of possible exposure to an infectious microbe. In some embodiments, a “subject” refers to on individual having or anticipated to have heterotopic ossification.

The term “diagnosed,” as used herein, refers to identifying and/or recognizing a disease (e.g., an infection or heterotopic ossification) by its signs and symptoms, or genetic analysis, pathological analysis, histological analysis, and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reactions that occur within a natural environment.

As used herein, the term “virulence” refers to the degree of pathogenicity of a microorganism, e.g., as indicated by the severity of the disease produced or its ability to invade the tissues of a subject. It is generally measured experimentally by the median lethal dose (LD50) or median infective dose (ID50). The term may also be used to refer to the competence of any infectious agent to produce pathologic effects.

As used herein, the term “effective amount” refers to the amount of a composition (e.g., a composition affecting the level or stability of ATP, dATP, a derivative or analog thereof, e.g., apyrase) sufficient to effect beneficial or desired results. An effective amount can be administered in one or more administrations, applications, or dosages and is not intended to be limited to a particular formulation or administration route.

As used herein the “minimum inhibitory concentration”, abbreviated “MIC”, is the lowest concentration of an antimicrobial agent that inhibits the growth (e.g., as detected by visual inspection) of a microorganism under certain conditions, e.g., after overnight incubation. MIC values are used to characterize the potency of a compound and a lower MIC value indicates a more potent compound.

As used herein, the term “IC₅₀” or “inhibition constant, 50%” refers to the lowest concentration of a compound that causes the activity of a biological molecule (e.g., an enzyme) to decrease to 50% of the activity of the biological molecule in the absence of the compound. IC₅₀ values are used to characterize the potency of a compound and a lower IC₅₀ value indicates a more potent compound.

As used herein, the terms “administration” and “administering” refer to the act of giving a drug, prodrug, or other agent, or therapeutic treatment (e.g., compositions of the present technology) to a physiological system (e.g., a subject or in vivo, in vitro, or ex vivo cells, tissues, and organs). Exemplary routes of administration to the human body are through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.), and the like.

As used herein, the term “treating a surface” refers to the act of exposing a surface to one or more compositions of the present technology. Methods of treating a surface include, but are not limited to, spraying, misting, submerging, and coating.

As used herein, the term “co-administration” refers to the administration of at least two agent(s) (e.g., an agent affecting the level or stability of ATP, dATP, a derivative or analog thereof′, or one such agent in combination with an antimicrobial agent) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration is readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent, e.g., as in when apyrase and an antimicrobial agent are co-administered.

As used herein, the term “wound” refers broadly to injuries to tissue including the skin, subcutaneous tissue, muscle, bone, and other structures initiated in different ways, for example, surgery, (e.g., open post-cancer resection wounds, including but not limited to, removal of melanoma and breast cancer, etc.), contained post-operative surgical wounds, pressure sores (e.g., from extended bed rest), wounds induced by trauma, and burns. As used herein, the term “wound” is used without limitation to the cause of the wound, e.g., a physical cause such as bodily positioning (e.g., as in bed sores) or an impact as with trauma, a chemical process such as a burn or exposure to a caustic chemical substance, or a biological cause such as a disease process, an aging process, an obstetric process, or any other manner of biological process. As used herein, “wound site” refers broadly to the anatomical location of a wound, without limitation.

As used herein, the term “dressing” refers broadly to any material applied to a wound for protection, absorbance, drainage, treatment, etc. Numerous types of dressings are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer) (Kannon and Garrett (1995) Dermatol. Surg. 21: 583-590; Davies (1983) Burns 10: 94; each herein incorporated by reference). The present technology also contemplates the use of dressings impregnated with pharmacological compounds (e.g., apyrase, antibiotics, antiseptics, thrombin, analgesic compounds, etc.). Cellular wound dressings include commercially available materials such as Apligraf®, Dermagraft®, Biobrane®, TransCyte®, Integra® Dermal Regeneration Template®, and OrCell®.

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., an antimicrobial or an agent affecting the level or stability of ATP, dATP, an analog or derivative thereof, e.g., apyrase) with a carrier, inert or active, making the composition especially suitable for diagnostic or therapeutic use in vitro, in vivo, or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present technology to the surface of the skin and mucosal cells and tissues (e.g., alveolar, buccal, lingual, masticatory, or nasal mucosa, and other tissues and cells which line hollow organs or body cavities).

As used herein, the term “pharmaceutically acceptable carrier” refers to any of the standard pharmaceutical carriers including, but not limited to, phosphate buffered saline, water, emulsions (e.g., such as an oil/water or water/oil emulsion), and various types of wetting agents, any and all solvents, dispersion media, coatings, sodium lauryl sulfate, isotonic and absorption delaying agents, disintegrants (e.g., potato starch or sodium starch glycolate), and the like. In some embodiments, the compositions include stabilizers and preservatives. For examples of carriers, stabilizers, and adjuvants, see, e.g., Martin, Remington's Pharmaceutical Sciences, 15th Ed., Mack Publ. Co. (Easton, Pa., 1975), incorporated herein by reference). In certain embodiments, the compositions of the present technology are formulated for veterinary, horticultural, or agricultural use. Such formulations include dips, sprays, seed dressings, stem injections, sprays, and mists. In certain embodiments, compositions of the present technology are used in any application where it is desirable to kill, inhibit, or prevent the growth and/or presence of a microbe, e.g., in food industry applications, consumer goods (e.g., medical goods, goods intended for consumers with impaired or developing immune systems (e.g., infants, children, elderly, consumers suffering from disease or at risk from disease)), and the like.

As used herein, the term “pharmaceutically acceptable salt” refers to any salt (e.g., obtained by reaction with an acid or a base) of a compound of the present technology that is physiologically tolerated in the target subject (e.g., a mammalian subject and/or in vivo or ex vivo, cells, tissues, or organs). “Salts” of the compounds of the present technology may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the technology and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW⁴⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present technology compounded with a suitable cation such as Na⁺, NH⁴⁺, and NW⁴⁺ (wherein W is a C₁₋₄ alkyl group), and the like. For therapeutic use, salts of the compounds of the present technology are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present technology are contemplated as being pharmaceutically acceptable. However, salts of acids and bases that are non-pharmaceutically acceptable may also find use, for example, in the preparation or purification of a pharmaceutically acceptable compound.

As used herein, the term “medical devices” includes any material or device that is used on, in, or through a subject's or patient's body, for example, in the course of medical treatment (e.g., for a disease or injury). Medical devices include, but are not limited to, such items as medical implants, wound care devices, drug delivery devices, and body cavity and personal protection devices. The medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, needles, drug delivery skin patches, drug delivery mucosal patches and medical sponges. Body cavity and personal protection devices, include, but are not limited to, tampons, sponges, surgical and examination gloves, contact lenses, and toothbrushes. Birth control devices include, but are not limited to, intrauterine devices (IUDs), diaphragms, and condoms.

As used herein, the term “therapeutic agent,” refers to compositions that decrease the infectivity, morbidity, or onset of mortality in a subject contacted by an infectious or pathogenic microbe or that prevent infectivity, morbidity, or onset of mortality in a host contacted by an infectious or pathogenic microbe. In some embodiments, the term “therapeutic agent,” refers to compositions that treat or prevent heterotopic ossification. As used herein, therapeutic agents encompass agents used prophylactically, e.g., in the absence of an infectious or pathogenic microbe or heterotopic ossification, in view of possible future exposure to an infectious or pathogenic microbe or possible future induction of heterotopic ossification. Such agents may additionally comprise pharmaceutically acceptable compounds (e.g., adjuvants, excipients, stabilizers, diluents, and the like). In some embodiments, the therapeutic agents of the present technology are administered in the form of topical compositions, injectable compositions, ingestible compositions, and the like. When the route is topical, the form may be, for example, a solution, cream, ointment, salve, or spray.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “cell culture” refers to any in vitro culture of cells, including, e.g., prokaryotic cells and eukaryotic cells. Included within this term are continuous cell lines (e.g., with an immortal phenotype), primary cell cultures, transformed cell lines, finite cell lines (e.g., non-transformed cells), bacterial or archaeal cultures in or on solid or liquid media, and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “kit” refers to any delivery system for delivering materials. In the context of reaction materials such as an agent that affects the level or stability of ATP, dATP, a derivative or analog thereof (e.g., apyrase), such delivery systems include but are not limited to systems that allow for the storage, transport, or delivery of appropriate reagents (e.g., apyrase, cells, buffers, culture media, selection reagents, etc., in the appropriate containers) and/or devices (e.g., catheters, syringes, reaction tubes or plates, culture tubes or plates) and/or supporting materials (e.g., media, written instructions for performing using the materials, etc.) from one location to another. For example, kits include one or more enclosures (e.g., boxes, bags) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain a dried composition (e.g., lyophilized apyrase, lyophilized dATP) with a gelling agent for a particular use, while a second container contains sterile fluid such as water or buffer for dissolving or re-suspending a dried composition.

The term “coating” as used herein refers to a layer of material covering, e.g., a medical device or a portion thereof. A coating can be applied to the surface or impregnated within the material of the implant.

As used herein, the term “antimicrobial agent” refers to composition that decreases, prevents, or inhibits the growth of bacterial, archaeal, and/or eukaryal organisms. Examples of antimicrobial agents include, e.g., antibiotics and antiseptics.

The term “antiseptic” as used herein is defined as an antimicrobial substance that inhibits the action of microorganisms, including but not limited to alpha-terpineol, methylisothiazolone, cetylpyridinium chloride, chloroxyleneol, hexachlorophene, chlorhexidine and other cationic biguanides, methylene chloride, iodine and iodophores, triclosan, taurinamides, nitrofurantoin, methenamine, aldehydes, azylic acid, silver, benzyl peroxide, alcohols, and carboxylic acids and salts. One skilled in the art is cognizant that these antiseptics can be used in combinations of two or more. Some examples of combinations of antiseptics include a mixture of chlorhexidine, chlorhexidine and chloroxylenol; chlorhexidine and methylisothiazolone; chlorhexidine and alpha-terpineol; methylisothiazolone and alpha-terpineol; thymol and chloroxylenol; chlorhexidine and cetylpyridinium chloride; or chlorhexidine, methylisothiazolone, and thymol. These combinations provide a broad spectrum of activity against a wide variety of organisms.

The term “antibiotics” as used herein is defined as a substance that inhibits the growth of microorganisms without substantial damage to the host. While some antibiotics may burden a host's physiological systems (e.g., processing and clearance by the liver or kidneys, digestive disruptions, allergic reactions), such phenomena are not intended to exclude known antibiotics from the scope of the term “antibiotics” as used herein. Examples of antibiotic activities include inhibiting cell wall synthesis, protein synthesis, nucleic acid synthesis, or altering cell membrane function.

Classes of antibiotics include, but are not limited to, macrolides (e.g., erythromycin), penicillins (e.g., nafcillin), cephalosporins (e.g., cefazolin), carbepenems (e.g., imipenem), monobactam (e.g., aztreonam), other beta-lactam antibiotics, beta-lactam inhibitors (e.g., sulbactam), oxalines (e.g. linezolid), aminoglycosides (e.g., gentamicin), chloramphenicol, sufonamides (e.g., sulfamethoxazole), glycopeptides (e.g., vancomycin), quinolones (e.g., ciprofloxacin), tetracyclines (e.g., minocycline), fusidic acid, trimethoprim, metronidazole, clindamycin, mupirocin, rifamycins (e.g., rifampin), streptogramins (e.g., quinupristin and dalfopristin) lipoprotein (e.g., daptomycin), polyenes (e.g., amphotericin B), azoles (e.g., fluconazole), and echinocandins (e.g., caspofungin acetate).

Examples of specific antibiotics include, but are not limited to, erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al, U.S. Pat. No. 4,642,104 herein incorporated by reference will readily suggest themselves to those of ordinary skill in the art.

As used herein, the term “protective agent” refers to a composition or compound that protects the activity or integrity of an active agent (e.g., an enzyme, e.g., apyrase) when the active agent is exposed to certain conditions (e.g., drying, freezing). Examples of protective agents include but are not limited to non-fat milk solids, trehalose, glycerol, betaine, sucrose, glucose, lactose, dextran, polyethylene glycol, sorbitol, mannitol, poly vinyl propylene, potassium glutamate, monosodium glutamate, Tween 20 detergent, Tween 80 detergent, and an amino acid hydrochloride.

As used herein, the term “gelling agent” refers to a composition that, when dissolved, suspended, or dispersed in a fluid (e.g., an aqueous fluid such as water or a buffer solution), forms a gelatinous semi-solid (e.g., a lubricant gel). Examples of gelling agents include but are not limited to hydroxyethyl cellulose, hydroxymethyl cellulose, hydroxypropyl guar, methyl cellulose, ethyl cellulose, hydroxypropyl cellulose, sodium carboxymethyl cellulose, carbomer, alginate, gelatin, and poloxamer.

As used herein, the term “excipient” refers to an inactive ingredient (i.e., not pharmaceutically active) added to a preparation of an active ingredient. The gelling and protective agents described herein are referred to generally as “excipients.”

### Aspects of the Technology

1. Apyrase

Apyrase (EC 3.6.1.5), also known as adenosine diphosphatase or, more generally, nucleotide phosphohydrolase, is a calcium- or magnesium-activated plasma membrane-bound glycoprotein enzyme that catalyzes the hydrolysis of adenosine triphosphate (ATP) to yield adenosine monophosphate (AMP) and inorganic phosphate (Pᵢ). Apyrase can also act on adenosine diphosphate (ADP) and other nucleoside triphosphates and diphosphates with the general reaction being:

NTP→NDP+Pᵢ→NMP+2Pᵢ

Apyrase is classified as an E-type ecto-ATPase because it has a relatively high substrate-specificity for extracellular ATP and ADP to yield AMP and inorganic phosphate and is has a low or no sensitivity to inhibitors of P-type, F-type, and V-type ATPases (Stout et al. (1994) “Purification and characterization of the ecto-Mg-ATPase of chicken gizzard smooth muscle” J Biochem Biophys Methods 29(1): 61-75; Lin et al. (1989) “Cloning and expression of a cDNA coding for a rat liver plasma membrane ecto-ATPase. The primary structure of the ecto-ATPase is similar to that of the human biliary glycoprotein I” J Biol Chem 264(24): 14408-14; Lin (1989) “Localization of the ecto-ATPase (ecto-nucleotidase) in the rat hepatocyte plasma membrane. Implications for the functions of the ecto-ATPase” J Biol Chem 264(24): 14403-7; Dombrowski et al. (1993) “Identification and partial characterization of an ectoATPase expressed by human natural killer cells” Biochemistry 32(26): 6515-22).

Biologically, apyrases are nonenergy-coupled NTPases that play diverse physiologic roles through their ability to change the ratios of energy carriers (e.g., ATP), inorganic phosphorus, and signaling molecules (e.g., GMP, cAMP). These basic activities are involved in diverse biological processes, e.g., in animals, apyrases are involved in neurotransmission and blood platelet aggregation; in yeasts, apyrases facilitate the glycosylation of N- and O-linked oligosaccharides in the Golgi lumen; and in plants, apyrases play a role in phosphate transport and mobilization.

Apyrase is ubiquitously found in eukaryotes mostly in membrane-bound forms but also in secreted soluble forms (e.g., the catalytic domain can be located cytoplasmically or extracellularly). Studies have shown that apyrase activities are present in a wide variety of organisms, tissues, and cell types such as rat hepatocytes, rabbit skeletal muscle, chicken gizzard smooth muscle, potato tubers, parasites such as Toxoplasma gondii and Cryptosporidium hominis, in mosquito saliva, and others. Two isoenzymes are found in commercial preparations from S. tuberosum. One isoenzyme has a 10× higher selectivity for ATP than for ADP and another isoenzyme that has no such selectivity (see, e.g., Lin (1989) “Localization of the ecto-ATPase (ecto-nucleotidase) in the rat hepatocyte plasma membrane. Implications for the functions of the ecto-ATPase” J Biol Chem 264(24): 14403-7; Treuheit et al. (1992) “Mg(2+)-ATPase from rabbit skeletal muscle transverse tubules is 67-kilodalton glycoprotein” J Biol Chem 267(17): 11777-82; Stout et al. (1994) “Purification and characterization of the ecto-Mg-ATPase of chicken gizzard smooth muscle” J Biochem Biophys Methods 29(1): 61-75; Handa et al. (1996) “Purification and cloning of a soluble ATP-diphosphohydrolase (apyrase) from potato tubers (Solanum tuberosum)” Biochem Biophys Res Commun 218(3): 916-23; Manque et al. (2012) “Identification and characterization of a novel calcium-activated apyrase from Cryptosporidium parasites and its potential role in pathogenesis” PLoS One 7(2): e31030; Bermudes et al. (1994) “Tandemly repeated genes encode nucleoside triphosphate hydrolase isoforms secreted into the parasitophorous vacuole of Toxoplasma gondii” J Biol Chem 269(46): 29252-60; Champagne et al. (1995) “The salivary gland-specific apyrase of the mosquito Aedes aegypti is a member of the 5′-nucleotidase family” Proc Natl Acad Sci USA 92(3): 694-8).

In humans, eight distinct types of apyrases and related enzymes exist with different substrate specificities and protein localizations that are implicated in diverse physiological functions (see Table 1). Of these, CD39L1 encoded by the gene ENTPD2 is an exemplary candidate for drug development to treat bacterial wound infections. CD39L1 exhibits a high substrate specificity for ATP (and low to moderate specificity for ADP) and is naturally expressed in many cell types, thus minimizing risks for provoking host immunological responses against therapeutic recombinant forms of CD39L1. Furthermore, the weak ADP hydrolyzing activities of CD39L1 provides for a controlled wound cicatrization and platelet aggregation by avoiding aggressive depletion of the ADP pool. In instances where wound cicatrization is not desirable, such as the case for burn wound injuries, a variant of CD39L1 that has improved ADP hydrolyzing activity can be readily engineered since CD39L1 is remarkably similar to CD39 and shares similar ADP binding domains.

TABLE 1
human ectonucleoside triphosphate diphosphohydrolases

| | | Preferred | | |
|---|---|---|---|---|
| Enzyme | Gene | Substrate | Localization | Description |
| CD39 | ENTPD1 | ATP, ADP | Membrane | Expressed in activated natural killer, lymphocyte B and T |
| | | Ratio (4:1) | | cells. Thought to protect immune cells from lysis, prevent |
| | | | | platelet aggregation by hydrolyzing ADP to AMP and |
| | | | | regulate purinergic neurotransmission in the nervous |
| | | | | system. |
| CD39L1 | ENTPD2 | ATP, ADP | Membrane | Expressed in various organs and tissues. Thought to |
| | | Ratio (11:1) | | regulate purinergic neurotransmission in the nervous |
| | | | | system. |
| CD39L2 | ENTPD6 | GDP, IDP | Golgi | Expressed in most tissues but primarily in the heart. |
| | | | | Hydrolyzes preferentially nucleoside diphosphates. |
| CD39L3 | ENTPD3 | ATP, ADP | Membrane | Expressed in adult brain, pancreas, spleen and prostate. |
| | | Ratio (4:1) | | |
| CD39L4 | ENTPD5 | UDP, GDP | Secreted | Expressed in adult liver, kidney, prostate, testis, and |
| | | | | colon. Poor expression in other tissues. Plays a key role |
| | | | | in the AKT1-PTEN signaling pathway by promoting |
| | | | | glycolysis in proliferating cells in response to (PI3K) |
| | | | | signaling. |
| LALP70 | ENTPD4 | UDP | Golgi | Ubiquitous expression, highest in testis but lowest in |
| | | | | bladder. Hydrolyzes preferentially nucleoside |
| | | | | diphosphates. |
| LALP1 | ENTPD7 | UTP, GTP | Vesicles | Preferentially hydrolyzes UTP and localizes inside |
| | | | | intracellular vesicles (referred as endo-apyrase). |
| SCAN1 | CANT1 | UDP, GDP | ER, Golgi | Widely expressed, poorly hydrolyzes ADP and ATP, |
| | | | | mutation results in Desbuquois dysplasia. |

2. Antimicrobial Agents

Embodiments of the technology relate to the surprising discovery that apyrase and antimicrobial agents act together synergistically. As demonstrated by the examples, apyrase increases the killing efficiency of antibacterial agents by 1000 times in some instances. As such, the technology provides therapies in which antimicrobial agents are administered in lower doses (e.g., 0.01%, 0.1%, 1%, 5%, 10%, 20%, 50%, 75%, 90%, etc. of the standard recommended dosage) than in conventional technologies. Importantly, this reduces toxic side effects, if present, of antimicrobial agents in subjects and helps to prevent drug resistance of microbes. In addition, the technology provided herein comprising combined use of apyrase and antimicrobial agents provides the only treatment to kill some multiply-resistant strains of pathogens.

In some embodiments, the antimicrobial agent is colistin. Colistin is a member of the polymyxin group of antibiotics and is known also as polymyxin E. Polymyxins are cationic polypeptides that disrupt the bacterial cell membrane through a detergent-like mechanism.

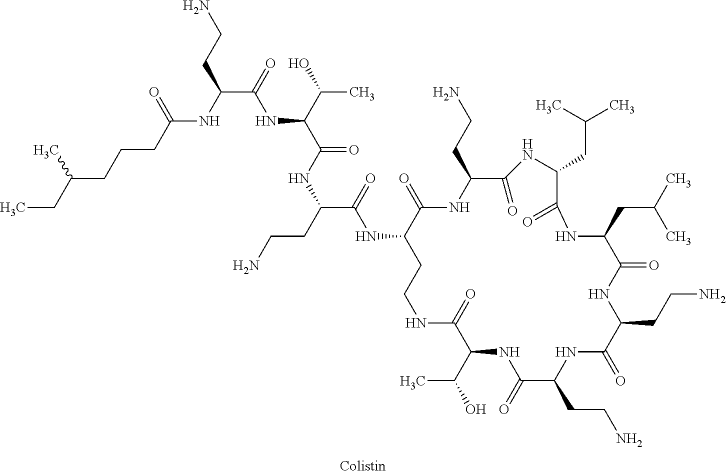 [SMILES: CCC(C)CCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]

This antimicrobial agent was first isolated in 1949 from Bacillus polymyxa var. colistinus and became available for clinical use in 1959. Colistin was given as an intramuscular injection for the treatment of gram-negative infections, but fell out of favor after aminoglycosides became available and thus provided drugs with fewer side effects. Colistin has been used as a topical therapy as part of selective digestive tract decontamination and is still used in aerosolized form for patients with cystic fibrosis. More recently, a number of centers around the world have used colistin intravenously for otherwise panresistant nosocomial infections, especially those due to Pseudomonas and Acinetobacter spp.

Research shows that colistin is a bactericidal drug that binds to lipopolysaccharides and phospholipids in the outer cell membrane of gram-negative bacteria. It competitively displaces divalent cations from the phosphate groups of membrane lipids, which leads to disruption of the outer cell membrane, leakage of intracellular contents, and bacterial death. In addition to its bactericidal effect, colistin binds and neutralizes lipopolysaccharide (LPS) and prevents the pathophysiologic effects of endotoxin in the circulation. It is to be understood that the technology does not depend on an understanding of the mechanism of action of colistin or of any antimicrobial agent and the technology is not limited in any way by any theory of mechanism of action. The technology may be practiced without any understanding and/or knowledge of the mode of action of the antimicrobial agent.

In some embodiments, the antimicrobial agent is an antimicrobial polymer, such as a peptide or a polymer referred to as E2 and E4 below:

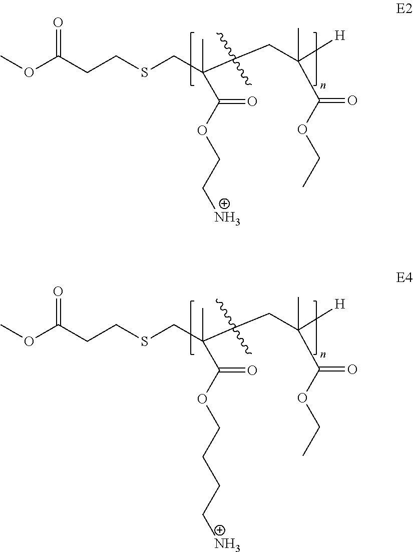

The bracketed portion of the structures comprising the NH₃+ is present in the polymer n number of times, e.g., from 1, 10, 50 to 100 or more times. Research has established the antimicrobial activity of these polymers. The MICs for E2 wherein n is 28 against Escherichia coli and Staphylococcus aureus are 86 μM and 43 μM, respectively, and the MICs for E4 wherein n is 29 against Escherichia coli and Staphylococcus aureus are 7.1 μM and 21 μM respectively.

The technology is not limited in the antimicrobial agent that is combined with apyrase, but is, in certain embodiments, any antimicrobial agent such as those discussed in this application. However, the technology is not limited to those discussed herein. Although the disclosure herein refers to certain illustrated embodiments, it is to be understood that these embodiments are presented by way of example and not by way of limitation.

3. Exemplary Microorganisms

While not limited to the killing of any particular microorgism, the technology has been shown to be effective against several clinically relevant pathogens. For example, several species of the genus Acinetobacter have clinical relevance as colonizing and/or pathogenic organisms. In particular, Acinetobacter baumannii is a pleomorphic aerobic gram-negative bacillus (similar in appearance to Haemophilus influenzae on Gram stain) commonly isolated from the hospital environment and hospitalized patients. A. baumannii is a water organism and preferentially colonizes aquatic environments. This organism is often cultured from hospitalized patients' sputum or respiratory secretions, wounds, and urine. In a hospital setting, Acinetobacter commonly colonizes irrigating solutions and intravenous solutions.

Acinetobacter species have low virulence but are capable of causing infection. Most Acinetobacter isolates recovered from hospitalized patients, particularly those recovered from respiratory secretions and urine, represent colonization rather than infection.

Acinetobacter infections (in contrast to colonizations) usually involve organ systems that have a high fluid content (e.g., respiratory tract, CSF, peritoneal fluid, urinary tract), manifesting as nosocomial pneumonia, infections associated with continuous ambulatory peritoneal dialysis (CAPD), or catheter-associated bacteriuria. The presence of Acinetobacter isolates in respiratory secretions in intubated patients nearly always represents colonization. Acinetobacter pneumonias occur in outbreaks and are usually associated with colonized respiratory-support equipment or fluids. Nosocomial meningitis may occur in colonized neurosurgical patients with external ventricular drainage tubes.

A. baumannii is a multiresistant aerobic gram-negative bacillus sensitive to relatively few antibiotics. Multidrug-resistant Acinetobacter is not a new or emerging phenomenon. Rather, A. baumannii has always been an organism inherently resistant to multiple antibiotics. Drugs to which A. baumannii is often susceptible include meropenem, colistin, polymyxin B, amikacin, rifampin, minocycline, and tigecycline. On the contrary, first-, second-, and third-generation cephalosporins, macrolides, and penicillins have little or no anti-Acinetobacter activity, and their use may actually predispose one to Acinetobacter colonization.

Other clinically relevant microorganisms and particular strains thereof (e.g., especially virulent and/or having particular drug resistances) are discussed in the examples, e.g., Pseudomonas aeruginosa, Burkholderia species, Escherichia coli, Staphylococcus aureus, Mycobacterium immunogenum, among others. The technology, however, is not limited to applications related to these organisms and these are provided as examples.

4. Pharmaceutical Formulations

In some embodiments, antimicrobial agents and agents affecting the level, incidence, or stability of dATP, ATP, analogs or derivatives thereof; e.g., apyrase, are preferably employed for therapeutic uses in combination with a suitable pharmaceutical carrier. Such compositions comprise an effective amount of the agents and a pharmaceutically acceptable carrier or excipient. The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the agents, some of which are described herein.

The term “agent” and “compound” are used herein interchangeably. Compounds may be in a formulation for administration topically, locally, or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, or micro spheres formed of biodegradable or non-biodegradable polymers or proteins or liposomes for targeting to cells. Such systems are well known to those skilled in the art and may be optimized for use with the appropriate agent.

In some embodiments, e.g., where agents described herein are used topically (e.g., on skin, at wound sites, at burn sites), the agent is preferably formulated for topical application. Formulations for topical administration may include ointments, lotions, creams, gels, drops, suppositories, sprays, liquids, and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampoules or in multi-dose containers, with an added preservative.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono- or di-glycerides. Aqueous carriers include water, alcoholic/aqueous solutions, emulsions or suspensions, including saline and buffered media. Parenteral vehicles include sodium chloride solution, 1,3-butanediol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The compounds alone or in combination with other suitable components can also be made into aerosol formulations (e.g., they can be nebulized) to be administered via inhalation. Aerosol formulations can be placed into pressurized acceptable propellants, such as dichlorodifluoromethane, propane, nitrogen, and the like. For administration by inhalation, the compounds are conveniently delivered in the form of an aerosol spray presentation from pressurized packs or a nebulizer, with the use of a suitable propellant. Similar aerosolized forms may be used for non-pharmaceutical applications, e.g., for spraying or coating inert surfaces.

In some embodiments, the compound described above may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In some embodiments, compounds are conjugated to lipophilic groups like cholesterol and lauric and lithocholic acid derivatives with C32 functionality to improve cellular uptake. Other groups that can be attached or conjugated to agents described herein to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines.

U.S. Pat. No. 6,919,208 to Levy, et al., herein incorporated by reference, also described methods for enhanced delivery. These pharmaceutical formulations may be manufactured in a manner that is itself known, e.g., by means of conventional mixing, dissolving, granulating, levigating, emulsifying, encapsulating, entrapping, or lyophilizing processes.

Compositions can be administered by a number of routes including, but not limited to: oral, intravenous, intraperitoneal, intramuscular, transdermal, subcutaneous, topical, sublingual, or rectal means. Compounds can also be administered via liposomes. Such administration routes and appropriate formulations are generally known to those of skill in the art.

Administration of the formulations described herein may be accomplished by any acceptable method which allows the compound(s) or agent(s) to reach their intended target.

The particular mode selected will depend, of course, upon factors such as the particular formulation, the severity of the state of the subject being treated, and the dosage required for therapeutic efficacy.

The actual effective amounts of compound can vary according to the specific compound or combination thereof being utilized, the particular composition formulated, the mode of administration, and the age, weight, condition of the individual, and severity of the symptoms or condition being treated.

Any acceptable method known to one of ordinary skill in the art may be used to administer a formulation to the subject. The administration may be localized (e.g., to a particular region, physiological system, tissue, organ, or cell type) or systemic, depending on the condition being treated.

Injections can be, e.g., intravenous, intradermal, subcutaneous, intramuscular, or intraperitoneal. The composition can be injected intradermally for treatment or prevention of an infection, for example. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The agents may be delivered in a manner that enables tissue-specific uptake of the agents and/or agent delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the agent over a certain time period, for example, hours, days, weeks, months, or years. This may be accomplished, for example, by repeated administrations of a formulation or by a sustained or controlled release delivery system in which the agent is delivered over a prolonged period without repeated administrations. Administration of the formulations using such a delivery system may be, for example, by oral dosage forms, bolus injections, transdermal patches, or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these.

Microcapsules of the foregoing polymers are described in, for example, U.S. Pat. No. 5,075,109, herein incorporated by reference. Other examples include nonpolymer systems that are lipid-based including sterols such as cholesterol, cholesterol esters, and fatty acids or neutral fats such as mono-, di-, and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which a composition is contained in a formulation within a matrix (for example, as described in U.S. Pat. Nos. 4,452,775, 4,675, 189, 5,736,152, 4,667,013, 4,748,034 and 5,239,660, herein incorporated by reference), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the composition. In addition, a pump-based hardware delivery system may be used to deliver one or more embodiments.

Examples of systems in which release occurs in bursts includes, e.g., systems in which the composition is entrapped in liposomes that are encapsulated in a polymer matrix, the liposomes being sensitive to specific stimuli, e.g., temperature, pH, light, and/or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition penetrates at a controlled rate, e.g., through a polymer. Such sustained release systems can be, e.g., in the form of pellets or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Dosages for a particular individual are determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to an individual is sufficient to effect a beneficial therapeutic response in the individual over time, or, e.g., to reduce symptoms, or other appropriate activity, depending on the application. The dose is determined by the efficacy of the particular formulation, and the activity, stability, or serum half-life of the composition employed and the condition of the individual, as well as the body weight or surface area of the individual to be treated. The size of the dose is also determined by the existence, nature, and extent of any adverse side-effects that accompany the administration of a particular compound, formulation, or the like in a particular individual. In non-pharmaceutical applications (e.g., treatment or coating of inert surfaces), the effective amount may similarly be determined by the stability of the composition employed and the condition, e.g., surface area or texture, to be treated, or the environment to which such surface is exposed.

Therapeutic compositions comprising one or more compounds are optionally tested in one or more appropriate in vitro and/or in vivo animal models of disease, to confirm efficacy, tissue metabolism, and to estimate dosages, according to methods well known in the art. In particular, dosages can be initially determined by activity, stability, or other suitable measures of treatment vs. non-treatment (e.g., comparison of treated vs. untreated cells or animal models), in a relevant assay. Formulations are administered at a rate determined by the LD50 of the relevant formulation and/or observation of any side-effects of the nucleic acids at various concentrations, e.g., as applied to the mass and overall health of the individual. Administration can be accomplished via single or divided doses.

In vitro models can be used to determine the effective doses of the compositions. In determining the effective amount of the compound to be administered in the treatment or prophylaxis of disease the physician evaluates circulating plasma levels, formulation toxicities, and progression of the disease or biological state.

The formulations described herein can supplement treatment conditions by any known conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. For example, compounds can also be administered in therapeutically effective amounts as a portion of an antibiotic, anti-infective, or anti-colonization cocktail.

5. Treatments

In some embodiments, a single dose of a composition according to the technology is administered to a subject. In other embodiments, multiple doses are administered over two or more time points, separated by hours, days, weeks, etc. In some embodiments, compounds are administered over a long period of time (e.g., chronically), for example, for a period of months or years (e.g., 1, 2, 3, 4, 5, 6, 7, 8, 9, 10, 11, 12, or more months or years). In such embodiments, compounds may be taken on a regular scheduled basis (e.g., daily, weekly, etc.) for the duration of the extended period.

Treatments include application to a living surface (e.g., including but not limited to skin, hair, teeth, fur, wound site, burn site, cells, tissues, organs, bodily fluids, blood, plasma, serum, cellular sample, acellular sample). Exemplary routes of administration (e.g., to a human body) include but are not limited to through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.), and the like. The technology should be broadly understood as associated with treatments of infections. As such, the technology includes embodiments in which apyrase is combined in a composition or treatment with any antimicrobial agent administered to control, inhibit, kill, contain, and/or debilitate a microbe and/or to decrease its virulence or effect on the subject.

The technology provided herein also includes kits for use in the instant methods. Kits of the technology comprise one or more containers comprising apyrase and an antimicrobial agent (e.g., in the same or in a different container), derivatives thereof, or pharmaceutically acceptable salts thereof, and/or a third agent, and in some variations further comprise instructions for use in accordance with any of the methods provided herein. The kit may further comprise a description of selecting an individual suitable treatment. Instructions supplied in the kits of the technology are typically written instructions on a label or package insert (e.g., a paper insert included with the kit), but machine-readable instructions (e.g., instructions carried on a magnetic or optical storage disk) are also contemplated. In some embodiments, the kit is a package containing a sealed container comprising any one of the preparations described above, together with instructions for use. The kit can also include a diluent container containing a pharmaceutically acceptable diluent. The kit can further comprise instructions for mixing the preparation and the diluent. The diluent can be any pharmaceutically acceptable diluent. Well known diluents include 5% dextrose solution and physiological saline solution. The container can be an infusion bag, a sealed bottle, a vial, a vial with a septum, an ampoule, an ampoule with a septum, an infusion bag, or a syringe. The containers can optionally include indicia indicating that the containers have been autoclaved or otherwise subjected to sterilization techniques. The kit can include instructions for administering the various solutions contained in the containers to subjects.

The technology also relates to methods of treatment with apyrase and (in some embodiments) an antibacterial agent. According to another aspect of the technology, a method is provided for treating a subject in need of such treatment with an effective amount of apyrase and (in some embodiments) an antibacterial agent or salts thereof. The method involves administering to the subject an effective amount of apyrase and (in some embodiments) an antibacterial agent or salts thereof in any one of the pharmaceutical preparations described above, detailed herein, and/or set forth in the claims. The subject can be any subject in need of such treatment. In the foregoing description, the technology is in connection with apyrase and (in some embodiments) an antibacterial agent or salts thereof. Such salts include, but are not limited to, bromide salts, chloride salts, iodide salts, carbonate salts, and sulfate salts. It should be understood, however, that apyrase and antibacterial agents are members of classes of compounds and the technology is intended to embrace pharmaceutical preparations, methods, and kits containing related derivatives within these classes. Another aspect of the technology then embraces the foregoing summary but read in each aspect as if any such derivative is substituted wherever “apyrase” or “antibacterial agent” or “antimicrobial agent” appears.

In some embodiments, a subject is tested to assess the presence, the absence, or the level of a disease (e.g., an infection or heterotopic ossification), e.g., by assaying or measuring a biomarker, a metabolite, a physical symptom, an indication, etc., to determine the risk of or the presence of an infection, and thereafter the subject is treated with apyrase and (in some embodiments) an antibacterial agent or salts thereof based on the outcome of the test. In some embodiments, a patient is tested, treated, and then tested again to monitor the response to therapy. In some embodiments, cycles of testing and treatment occur without limitation to the pattern of testing and treating (e.g., test/treat, test/treat/test, test/treat/test/treat, test/treat/test/treat/test, test/treat/treat/test/treat/treat, etc.), the periodicity, or the duration of the interval between each testing and treatment phase.

6. Other Uses and Treatments

In some embodiments, apyrase is used to treat heterotopic ossification. Heterotopic ossification (HO) is a musculoskeletal disease that is characterized by the formation of mature bone in soft tissues such as muscle, tendon, or fascia and is a frequent complication following trauma, burns, upper extremity injuries, or surgeries. Over 60% of severe burn patients will develop HO in at least one joint during recovery. Additionally, HO can occur secondary to other conditions, including soft-tissue trauma, amputation, central nervous system injury (e.g., traumatic brain injuries, spinal cord lesions, tumors, encephalitis), vasculopathies, arthroplasties (e.g., total hip arthroplasty), and end-stage cardiac valve disease.

The pathogenesis of heterotopic ossification remains unclear, though the inciting event is thought to be inflammation caused by trauma, surgery, or burns. This inflammation stimulates the recruitment of mesenchymal stem cells (MSCs) as well as endochondral ossification of resident MSCs. Current treatment and prophylactic strategies are not effective to prevent HO formation and/or to treat and reconstruct joints once HO has developed. Conventional technologies for treatment involve surgical extirpation of the heterotopic bone. However, even after a technically successful operation, over 75% of patients have difficulty maintaining their range of motion, 35% of patients have residual bone, over 10% of patients recur, and even those patients without complications suffer from significant joint contractures. In the face of such staggering numbers and suboptimal treatments, a substantial need exists for the development of methods to prevent HO. Thus, a significant clinical need exists to be able to prevent the formation of heterotopic ossification.

Accordingly, provided herein is technology related to treating and preventing heterotopic bone formation by inhibiting inflammation at injury sites with apyrase. While the technology is not limited by any theory and an understanding of the mechanism underlying the technology is not required to practice the technology, recent data demonstrates the role of bone morphogenic protein (BMP) signaling in mesenchymal stem cell (MSC) osteogenesis. In addition, it is contemplated that the massive inflammatory response to injury, such as a burn, enhances the osteogenic capacity of MSC niches and that this osteogenic potential, through the BMP-2 pathway, can be mitigated through ATP inhibition at the burn site, e.g., by apyrase.

As some subjects, such as burn and trauma victims, are often also susceptible to infections by pathogens, the technology described herein provides a single treatment for both treating and preventing infection (e.g., by a drug resistant pathogen) while simultaneously treating and preventing heterotopic ossification.

### EXAMPLES

### Example 1

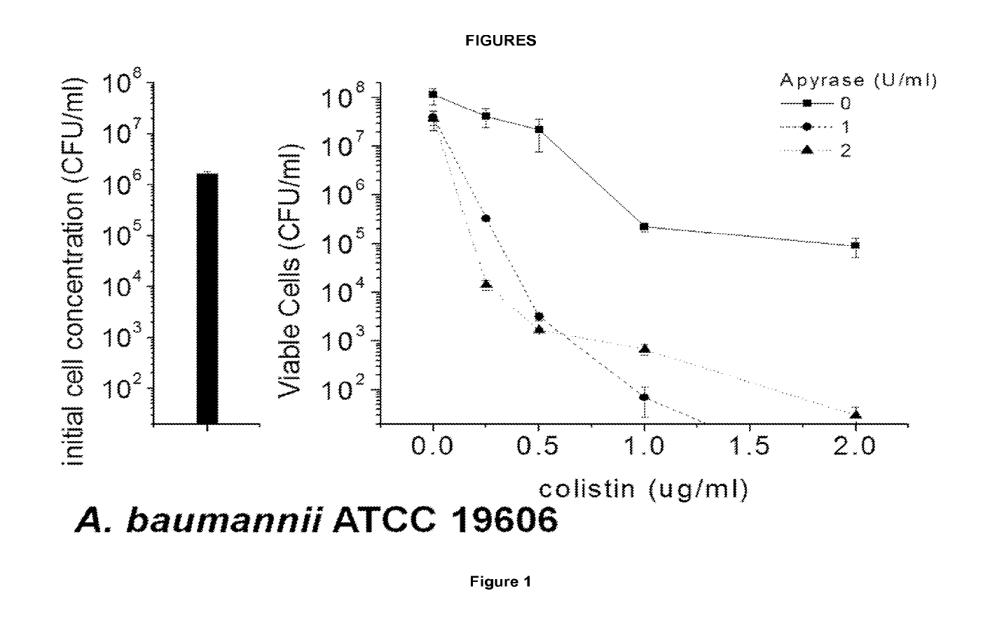

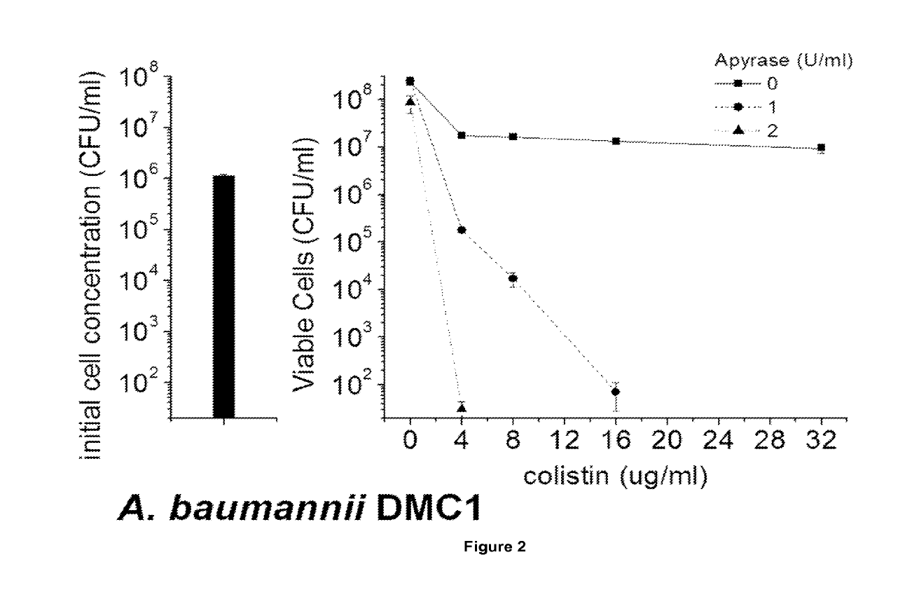

During the development of embodiments of the technology provided herein, data were collected showing that apyrase increases the effectiveness of antimicrobial agents. Approximately 10⁶ CFU/ml of A. baumannii strain ATCC 19606 was grown on solid medium culture plates in the presence of combinations of 0, 1, and 2 U/ml of apyrase and 0, 0.25, 0.5, 1, and 2 μg/ml of colistin. After incubation, the numbers of viable cells were determined by counting the colony forming units (CFU) on the plate (data provided as CFU/ml). FIG. 1 shows that apyrase improved the efficiency of killing A. baumannii strain ATCC 19606 by colistin. As shown in FIGS. 1, 1 and 2 U/ml of apyrase reduced the viable cell count to below 10 to 100 CFU/ml while approximately 10⁵ CFU/ml remained in samples treated with colistin alone at the some concentrations. In these Examples, the colistin is colistin sulfate salt, catalog number C4461 from Sigma-Aldrich (St. Louis, Mo.) and the apyrase is catalog number M0393L from New England BioLabs Inc. (Ipswich, Mass.).

### Example 2

During the development of embodiments of the technology provided herein, data were collected showing that apyrase increases the effectiveness of antimicrobial agents.

Approximately 10⁶ CFU/ml of A. baumannii strain DMC1 was grown on solid medium culture plates in the presence of combinations of 0, 1, and 2 U/ml of apyrase and 0, 4, 8, 16, and 32 μg/ml of colistin. After incubation, the numbers of viable cells were determined by counting the colony forming units (CFU) on the plate (data provided as CFU/ml). FIG. 2 shows that apyrase improved the efficiency of killing A. baumannii strain DMC1 by colistin. As shown in FIG. 2, 1 and 2 U/ml of apyrase reduced the viable cell count to below 10 to 100 CFU/ml while approximately 10⁷ CFU/ml remained in samples treated with colistin alone at the same concentrations.

### Example 3

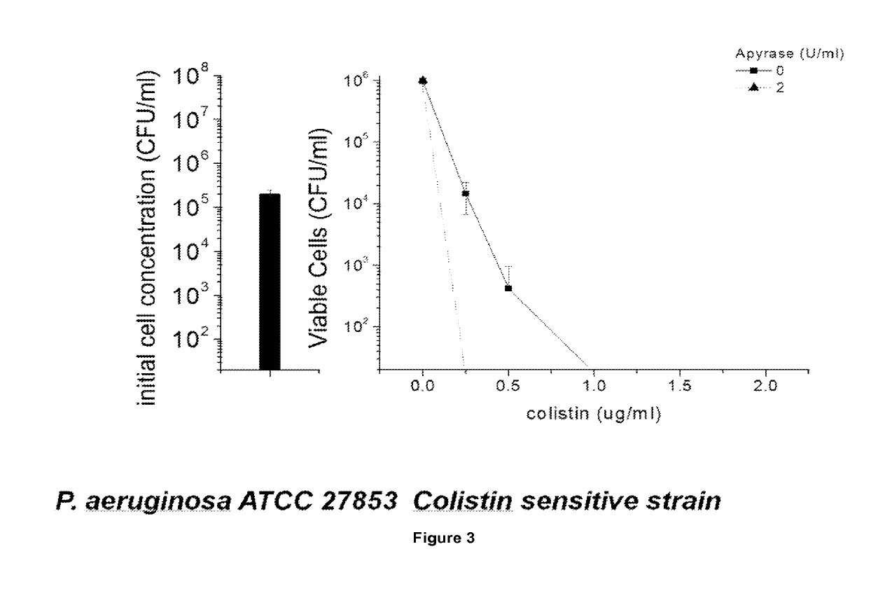

During the development of embodiments of the technology provided herein, data were collected showing that apyrase increases the effectiveness of antimicrobial agents on an antibiotic sensitive strain. Approximately 10⁵ to 10⁶ CFU/ml of colistin sensitive P. aeruginosa strain ATCC 27853 was grown in the presence of combinations of 0 and 2 U/ml of apyrase and 0, 0.25, and 0.5 μg/ml of colistin. After incubation, the numbers of viable cells were determined by counting the colony forming units (CFU) on the plate (data provided as CFU/ml). FIG. 3 shows that apyrase improved the efficiency of killing P. aeruginosa strain ATCC 27853 by colistin.

### Example 4

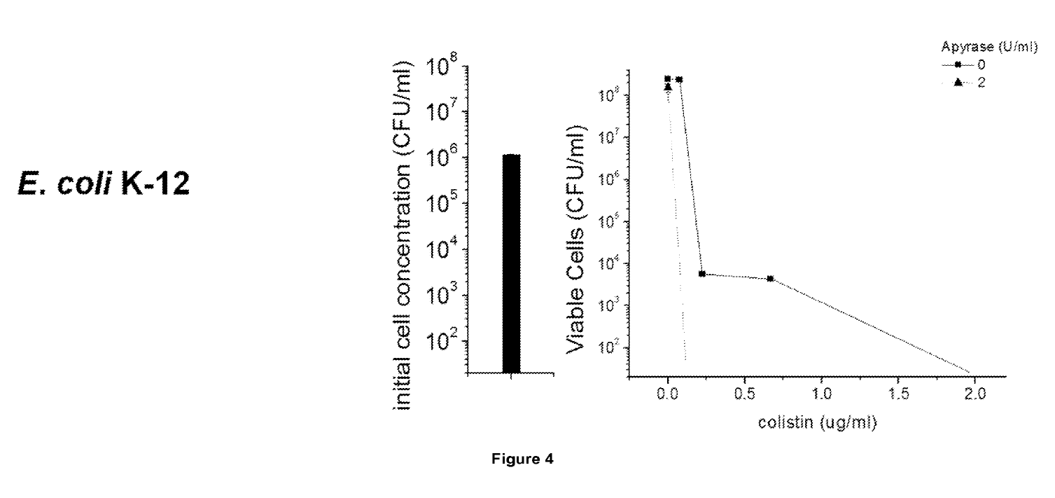

During the development of embodiments of the technology provided herein, data were collected showing that apyrase increases the effectiveness of antimicrobial agents. Approximately 10⁶ CFU/ml of E. coli strain K12 was grown in the presence of combinations of 0 and 2 U/ml of apyrase and from approximately 0 to 2 μg/ml of colistin. After incubation, the numbers of viable cells were determined by counting the colony forming units (CFU) on the plate (data provided as CFU/ml). FIG. 4 shows that apyrase improved the efficiency of killing E. coli strain K12 by colistin.

### Example 5

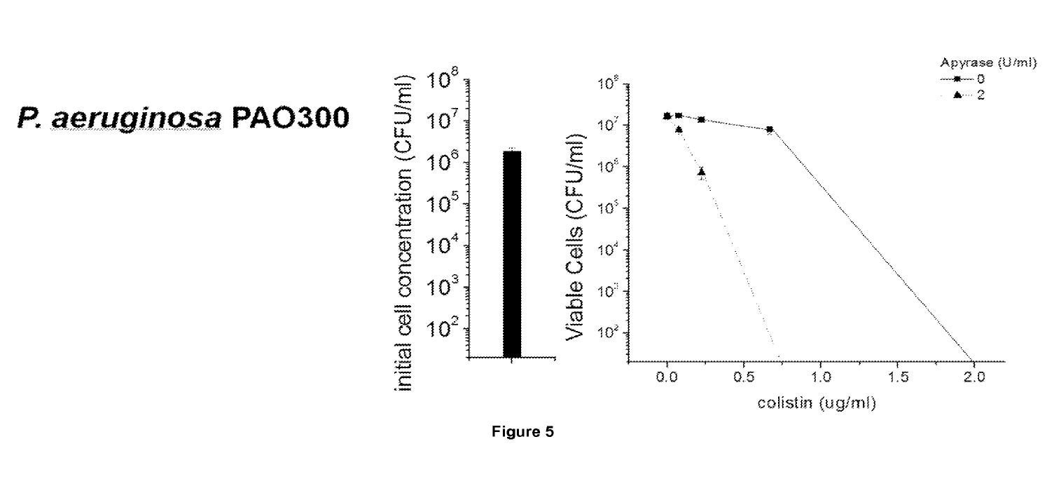

During the development of embodiments of the technology provided herein, data were collected showing that apyrase increases the effectiveness of antimicrobial agents. Approximately 10⁶ to 10⁷ CFU/ml of P. aeruginosa strain PAO300 was grown in the presence of combinations of 0 and 2 U/ml of apyrase and approximately 0 to 2 μg/ml of colistin. After incubation, the numbers of viable cells were determined by counting the colony forming units (CFU) on the plate (data provided as CFU/ml). FIG. 5 shows that apyrase improved the efficiency of killing P. aeruginosa strain PAO300 by colistin.

### Example 6

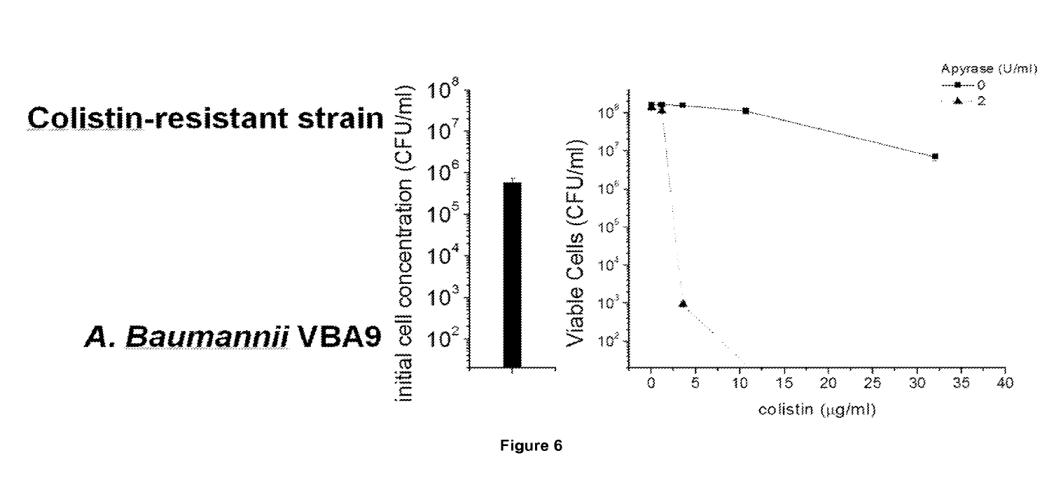

During the development of embodiments of the technology provided herein, data were collected showing that apyrase increases the effectiveness of antimicrobial agents against antibiotic resistant strains. Approximately 10⁵ to 10⁶ CFU/ml of the colistin-resistant A. baumannii strain VBA9 was grown in the presence of combinations of 0 and 2 U/ml of apyrase and approximately 0 to 32 μg/ml of colistin. After incubation, the numbers of viable cells were determined by counting the colony forming units (CFU) on the plate (data provided as CFU/ml). FIG. 6 shows that apyrase improved the efficiency of killing A. baumannii strain VBA9 by colistin. Apyrase increased the effectiveness of colistin at less than 10-15 μg/ml such that less than 10 to 100 CFU/ml were detected relative to greater than 10⁶ to 10⁷ in the presence of colistin alone.

### Example 7

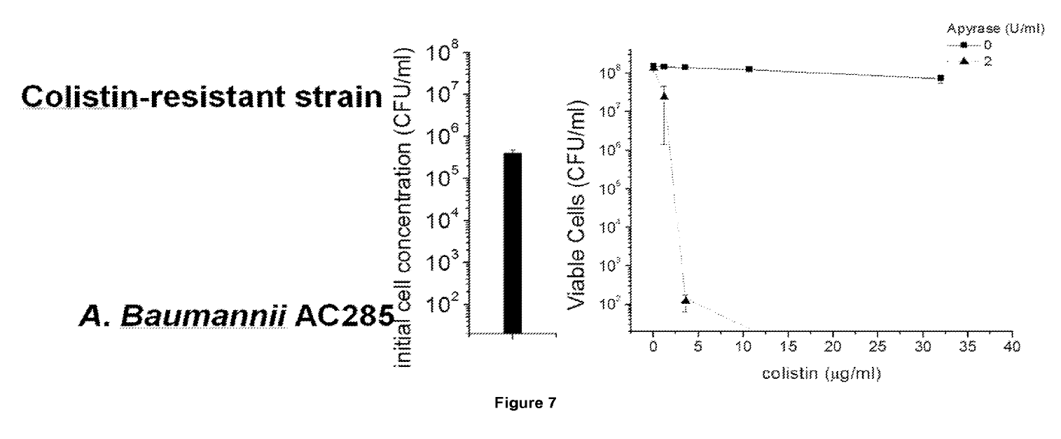

During the development of embodiments of the technology provided herein, data were collected showing that apyrase increases the effectiveness of antimicrobial agents against antibiotic resistant strains. Approximately 10⁵ to 10⁶ CFU/ml of the colistin-resistant A. baumannii strain AC285 was grown in the presence of combinations of 0 and 2 U/ml of apyrase and approximately 0 to 32 μg/ml of colistin. After incubation, the numbers of viable cells were determined by counting the colony forming units (CFU) on the plate (data provided as CFU/ml). FIG. 7 shows that apyrase improved the efficiency of killing A. baumannii strain AC285 by colistin. Apyrase increased the effectiveness of colistin at less than 5-10 μg/ml such that less than 10 to 100 CFU/ml were detected relative to greater than 10⁷ to 10⁸ in the presence of colistin alone at the same concentrations.

### Example 8

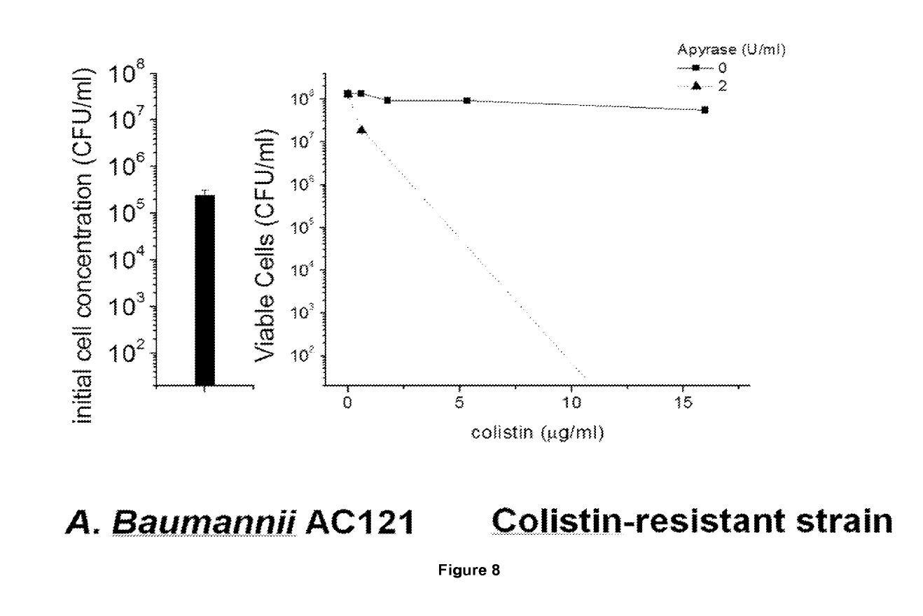

During the development of embodiments of the technology provided herein, data were collected showing that apyrase increases the effectiveness of antimicrobial agents against antibiotic resistant strains. Approximately 10⁵ to 10⁶ CFU/ml of the colistin-resistant A. baumannii strain AC121 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 8 shows that apyrase improved the efficiency of killing A. baumannii strain AC121 by colistin. Apyrase increased the effectiveness of colistin at less than 20 μg/ml such that less than 10 to 100 CFU/ml were detected relative to greater than 10⁷ to 10⁸ in the presence of colistin alone at the same concentrations.

### Example 9

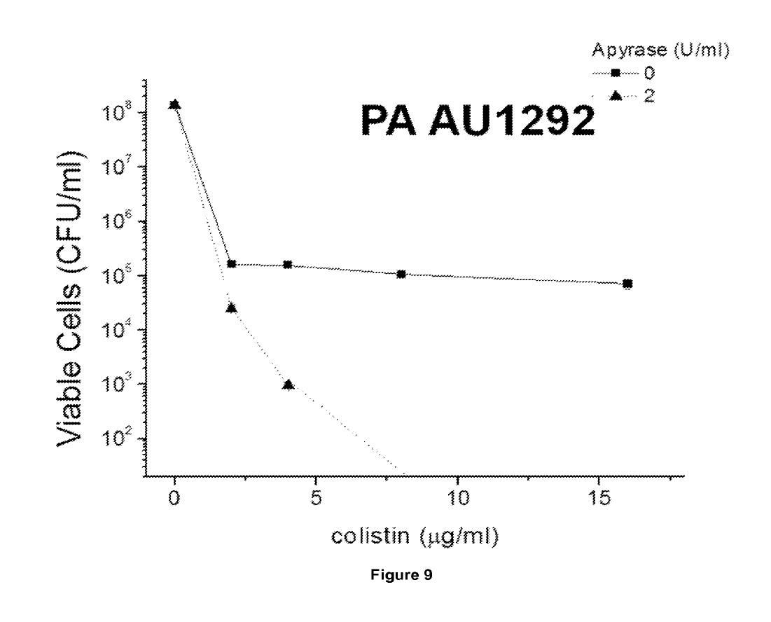

During the development of embodiments of the technology provided herein, data were collected showing that apyrase increases the effectiveness of antimicrobial agents. P. aeruginosa strain AU1292 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 9 shows that apyrase improved the efficiency of killing P. aeruginosa strain AU1292 by colistin. Apyrase increased the effectiveness of colistin at less than 20 μg/ml such that less than 10 to 100 CFU/ml were detected relative to greater than 10⁴ to 10⁵ in the presence of colistin alone at the same concentrations.

### Example 10

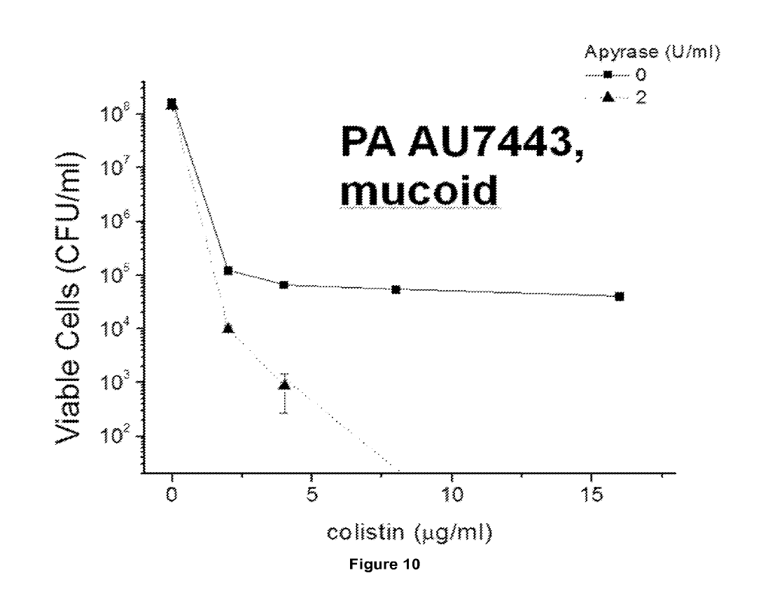

During the development of embodiments of the technology provided herein, data were collected showing that apyrase increases the effectiveness of antimicrobial agents. P. aeruginosa strain AU7443, mucoid was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 10 shows that apyrase improved the efficiency of killing P. aeruginosa strain AU7443, mucoid by colistin. Apyrase increased the effectiveness of colistin at less than 20 μg/ml such that less than 10 to 100 CFU/ml were detected relative to greater than 10⁴ to 10⁵ in the presence of colistin alone at the same concentrations.

### Example 11

During the development of embodiments of the technology provided herein, data were collected showing that apyrase increases the effectiveness of antimicrobial agents.

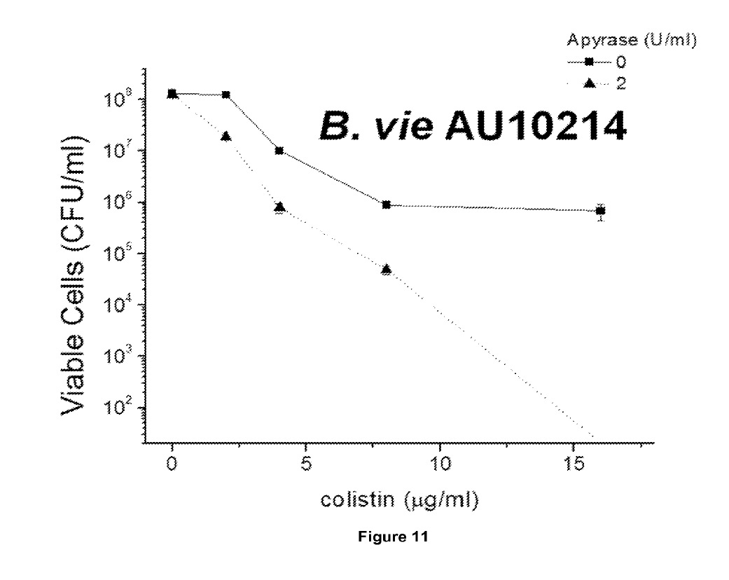

Burkholderia vietnamiensis AU10214 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 11 shows that apyrase improved the efficiency of killing Burkholderia vietnamiensis AU10214 by colistin. Apyrase increased the effectiveness of colistin at less than 20 μg/ml such that less than 10 to 100 CFU/ml were detected relative to greater than 10⁵ to 10⁶ in the presence of colistin alone at the same concentrations.

### Example 12

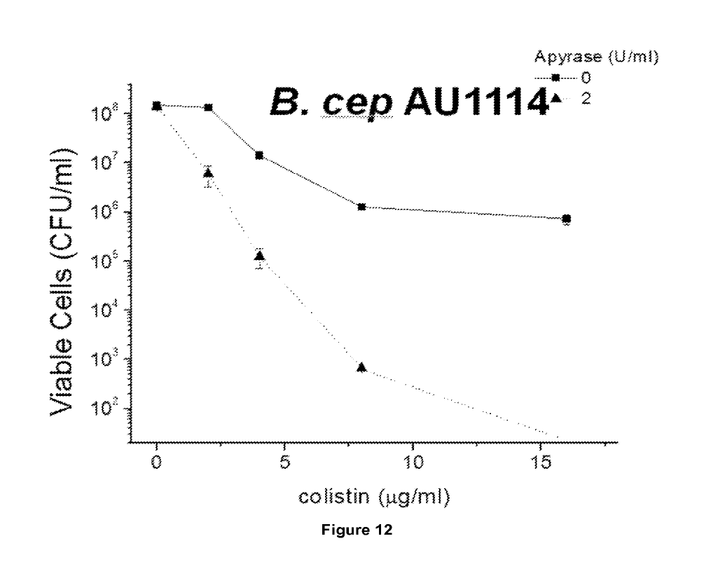

During the development of embodiments of the technology provided herein, data were collected showing that apyrase increases the effectiveness of antimicrobial agents. Burkholderia cepacia AU1114 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 12 shows that apyrase improved the efficiency of killing Burkholderia cepacia AU1114 by colistin. Apyrase increased the effectiveness of colistin at less than 20 μg/ml such that less than 10 to 100 CFU/ml were detected relative to greater than 10⁵ to 10⁶ in the presence of colistin alone at the same concentrations.

### Example 13

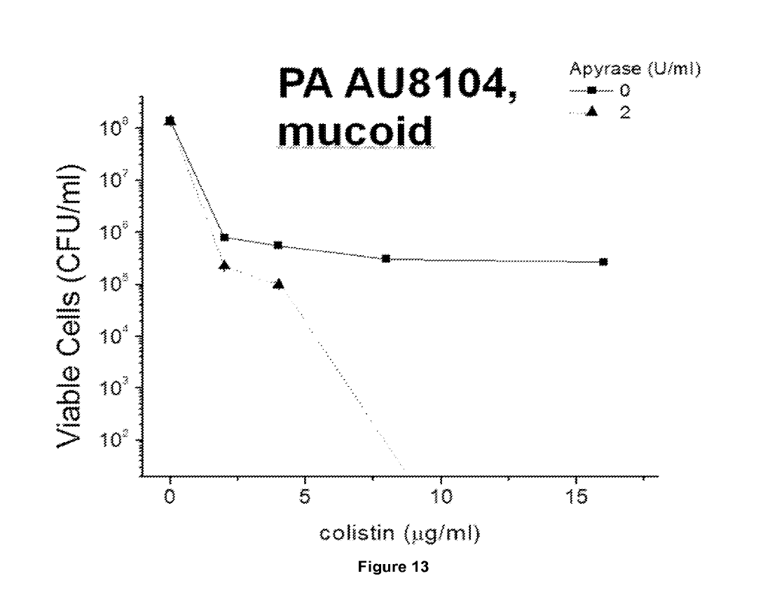

During the development of embodiments of the technology provided herein, data were collected showing that apyrase increases the effectiveness of antimicrobial agents. P. aeruginosa strain AU8104 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 13 shows that apyrase improved the efficiency of killing P. aeruginosa strain AU8104 by colistin. Apyrase increased the effectiveness of colistin at less than 10 μg/ml such that less than 10 to 100 CFU/ml were detected relative to greater than 10⁵ to 10⁶ in the presence of colistin alone at the same concentrations.

### Example 14

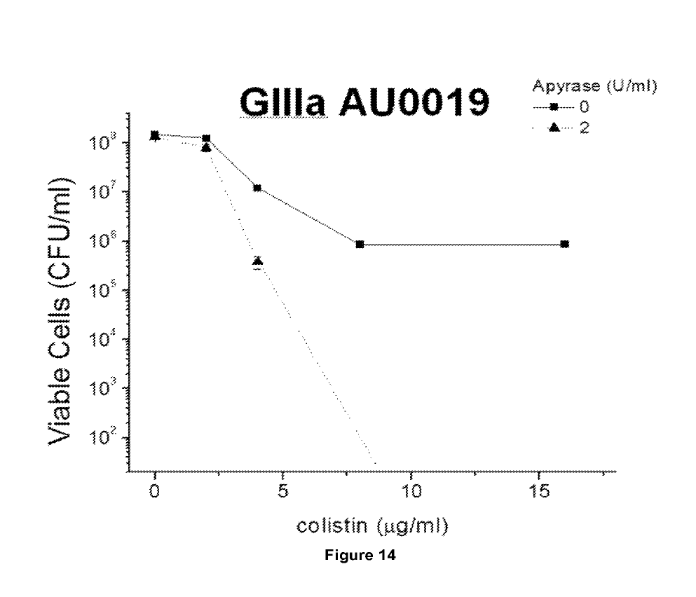

During the development of embodiments of the technology provided herein, data were collected showing that apyrase increases the effectiveness of antimicrobial agents. Burkholderia cepacia GIIIa AU0019 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 14 shows that apyrase improved the efficiency of killing Burkholderia cepacia GIIIa AU0019 by colistin. Apyrase increased the effectiveness of colistin at less than 10 μg/ml such that less than 10 to 100 CFU/ml were detected relative to greater than 10⁵ to 10⁶ in the presence of colistin alone at the same concentrations.

### Example 15

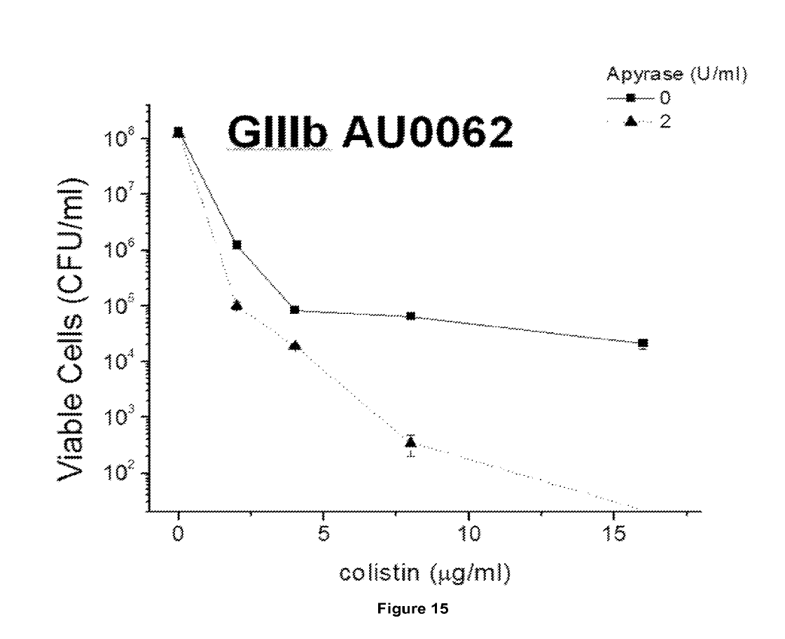

During the development of embodiments of the technology provided herein, data were collected showing that apyrase increases the effectiveness of antimicrobial agents. Burkholderia cepacia GIIIb AU0062 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 15 shows that apyrase improved the efficiency of killing Burkholderia cepacia GIIIb AU0062 by colistin. Apyrase increased the effectiveness of colistin at less than 20 μg/ml such that less than 10 to 100 CFU/ml were detected relative to greater than 10⁴ to 10⁵ in the presence of colistin alone at the same concentrations.

### Example 16

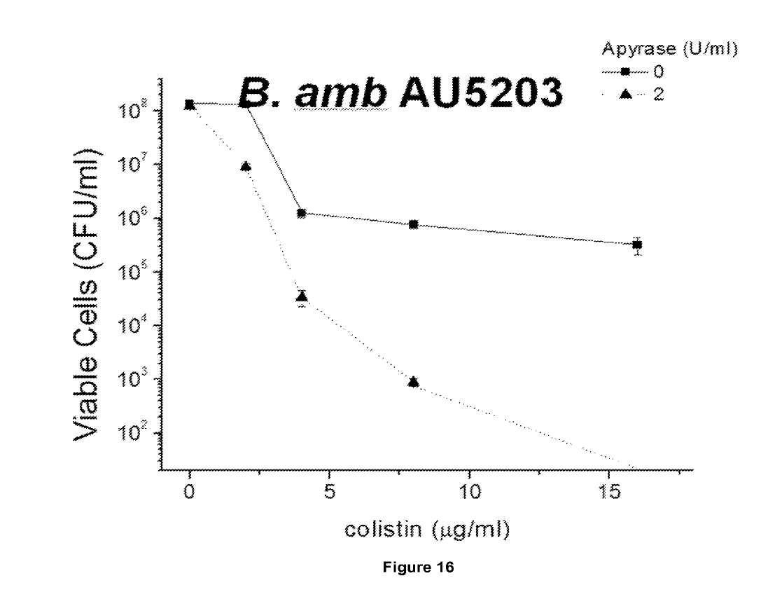

During the development of embodiments of the technology provided herein, data were collected showing that apyrase increases the effectiveness of antimicrobial agents. Burkholderia ambifaria AU5203 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 16 shows that apyrase improved the efficiency of killing Burkholderia ambifaria AU5203 by colistin. Apyrase increased the effectiveness of colistin at less than 20 μg/ml such that less than 10 to 100 CFU/ml were detected relative to greater than 10⁵ to 10⁶ in the presence of colistin alone at the same concentrations.

### Example 17

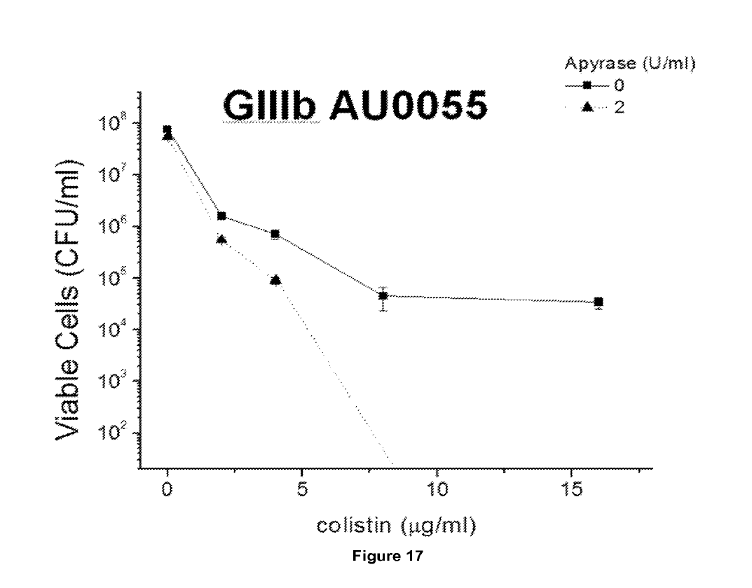

During the development of embodiments of the technology provided herein, data were collected showing that apyrase increases the effectiveness of antimicrobial agents. Burkholderia cepacia GIIIb AU0055 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 17 shows that apyrase improved the efficiency of killing Burkholderia cepacia GIIIb AU0055 by colistin. Apyrase increased the effectiveness of colistin at less than 10 μg/ml such that less than 10 to 100 CFU/ml were detected relative to greater than 10⁴ to 10⁵ in the presence of colistin alone at the same concentrations.

### Example 18

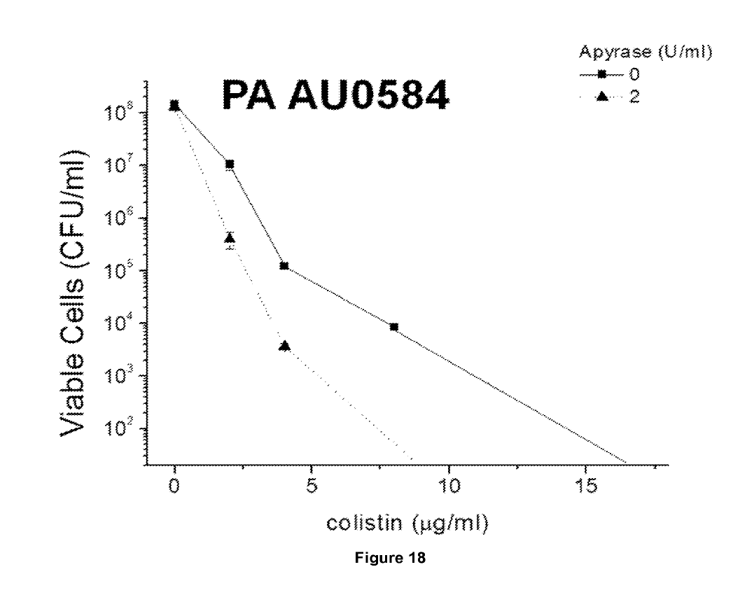

During the development of embodiments of the technology provided herein, data were collected showing that apyrase increases the effectiveness of antimicrobial agents. P. aeruginosa AU0584 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 18 shows that apyrase improved the efficiency of killing P. aeruginosa AU0584 by colistin. Apyrase increased the effectiveness of colistin at less than 10 μg/ml such that less than 10 to 100 CFU/ml were detected relative to greater than 10³ to 10⁴ in the presence of colistin alone at the same concentrations.

### Example 19

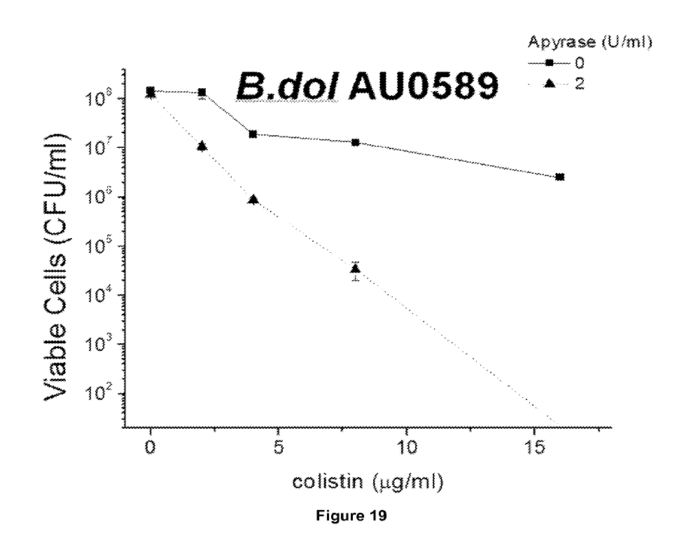

During the development of embodiments of the technology provided herein, data were collected showing that apyrase increases the effectiveness of antimicrobial agents. Burkholderia dolosa AU0589 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 19 shows that apyrase improved the efficiency of killing Burkholderia dolosa AU0589 by colistin. Apyrase increased the effectiveness of colistin at less than 20 μg/ml such that less than 10 to 100 CFU/ml were detected relative to greater than 10⁶ to 10⁷ in the presence of colistin alone at the same concentrations.

### Example 20

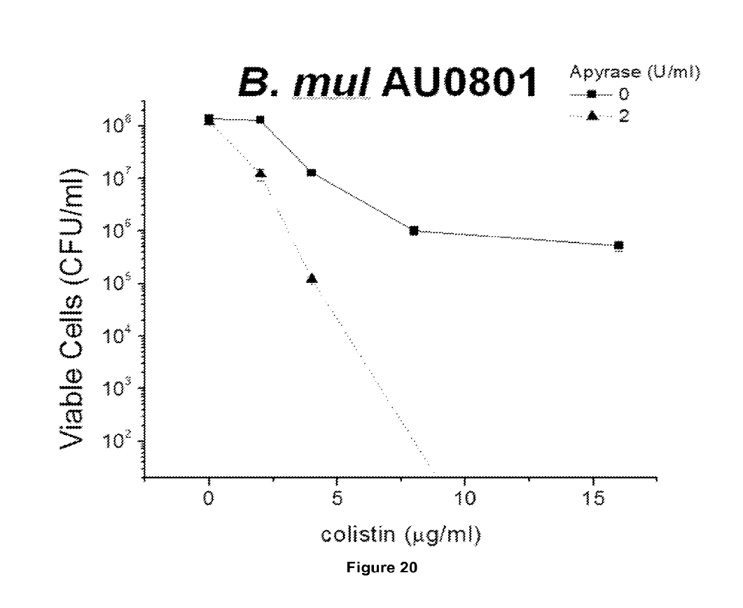

During the development of embodiments of the technology provided herein, data were collected showing that apyrase increases the effectiveness of antimicrobial agents. Burkholderia multivorans AU0801 was grown in the presence of combinations of 0 and 2 U/ml of apyrase and approximately 0 to 20 μg/ml of colistin. After incubation, the numbers of viable cells were determined by counting the colony forming units (CFU) on the plate (data provided as CFU/ml). FIG. 20 shows that apyrase improved the efficiency of killing Burkholderia multivorans AU0801 by colistin. Apyrase increased the effectiveness of colistin at less than 10 μg/ml such that less than 10 to 100 CFU/ml were detected relative to greater than 10⁵ to 10⁶ in the presence of colistin alone at the same concentrations.

### Example 21

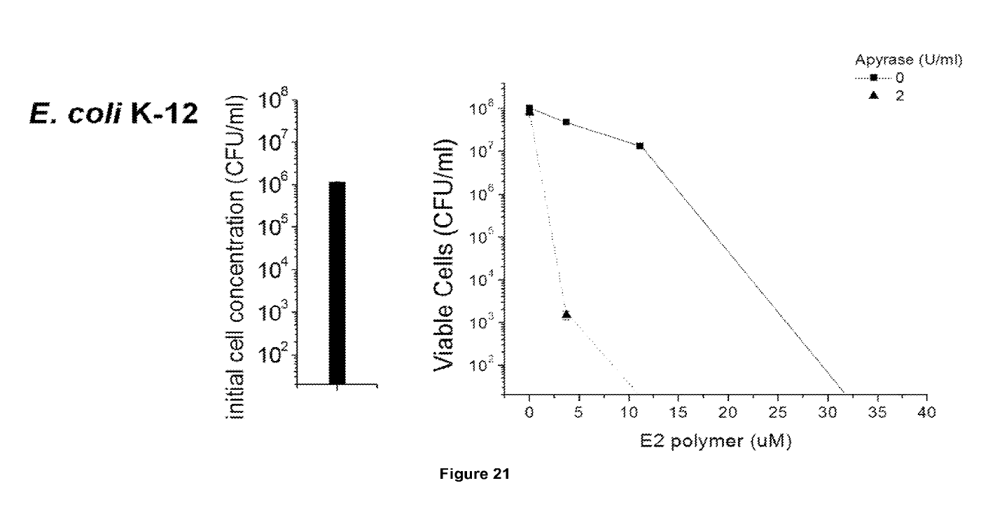

During the development of embodiments of the technology provided herein, data were collected showing that apyrase increases the effectiveness of antimicrobial agents. Approximately 10⁶ CFU/ml of E. coli K12 was grown in the presence of combinations of 0 and 2 U/ml of apyrase and approximately 0 to 40 μM of antibacterial polymer E2. After incubation, the numbers of viable cells were determined by counting the colony forming units (CFU) on the plate (data provided as CFU/ml). FIG. 21 shows that apyrase improved the efficiency of killing E. coli K12 by E2. Apyrase increased the effectiveness of E2 polymer at less than 15 μM such that less than 10 to 100 CFU/ml were detected relative to greater than 10⁷ to 10⁸ in the presence of E2 polymer alone at the same concentrations.

### Example 22

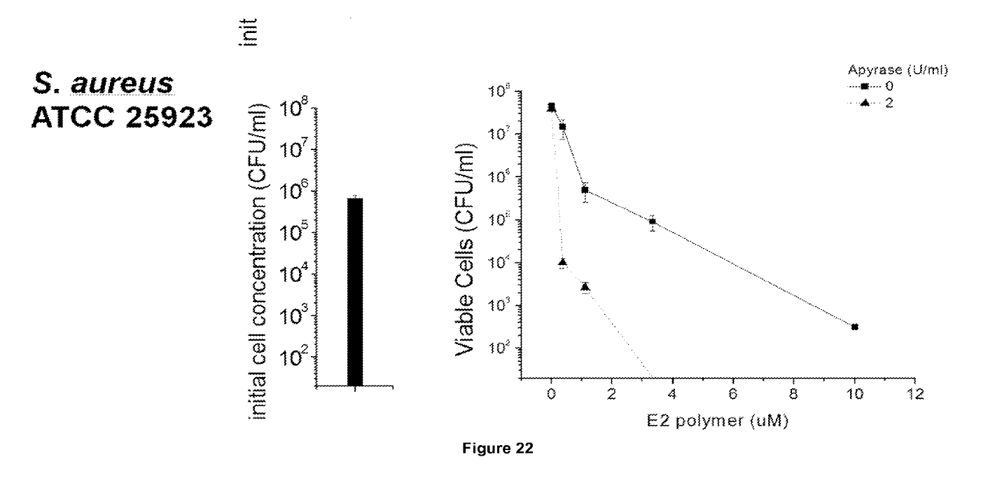

During the development of embodiments of the technology provided herein, data were collected showing that apyrase increases the effectiveness of antimicrobial agents. Approximately 10⁵ to 10⁶ CFU/ml of S. aureus ATCC 25923 was grown in the presence of combinations of 0 and 2 U/ml of apyrase and approximately 0 to 12 μM of antibacterial polymer E2. After incubation, the numbers of viable cells were determined by counting the colony forming units (CFU) on the plate (data provided as CFU/ml). FIG. 22 shows that apyrase improved the efficiency of killing S. aureus ATCC 25923 by polymer E2. Apyrase increased the effectiveness of E2 polymer at less than 4 μM such that less than 10 to 100 CFU/ml were detected relative to greater than 10⁵ to 10⁶ in the presence of E2 polymer alone at the same concentrations.

### Example 23

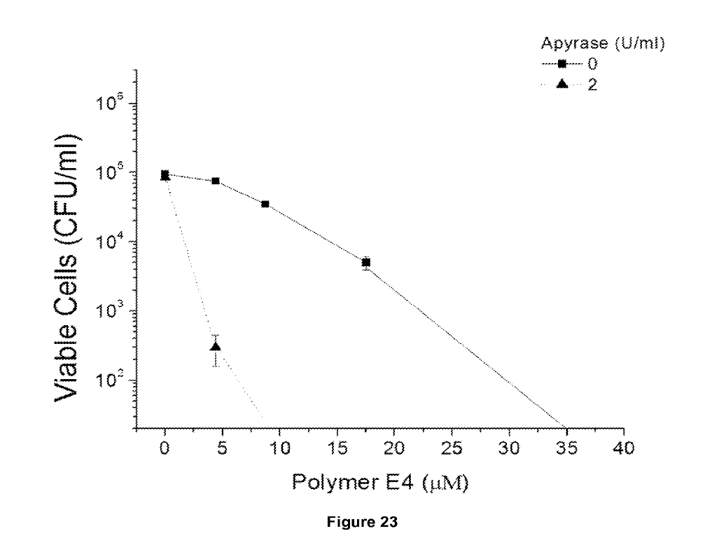

During the development of embodiments of the technology provided herein, data were collected showing that apyrase increases the effectiveness of antimicrobial agents. Approximately 10⁴ to 10⁵ CFU/ml of Mycobacterium immunogenum ATCC 700505 was grown in 7H9 medium for 4 hours at 37° C. in the presence of combinations of 0 and 2 U/ml of apyrase and approximately 0 to 40 μM of antibacterial polymer E4. After incubation, the numbers of viable cells were determined by counting the colony forming units (CFU) on the plate (data provided as CFU/ml). FIG. 23 shows that apyrase improved the efficiency of killing Mycobacterium immunogenum ATCC 700505 by polymer E4. Apyrase increased the effectiveness of E4 polymer at less than 10 μM such that less than 10 to 100 CFU/ml were detected relative to greater than 10⁴ to 10⁵ in the presence of E4 polymer alone at the same concentrations.

### Example 24

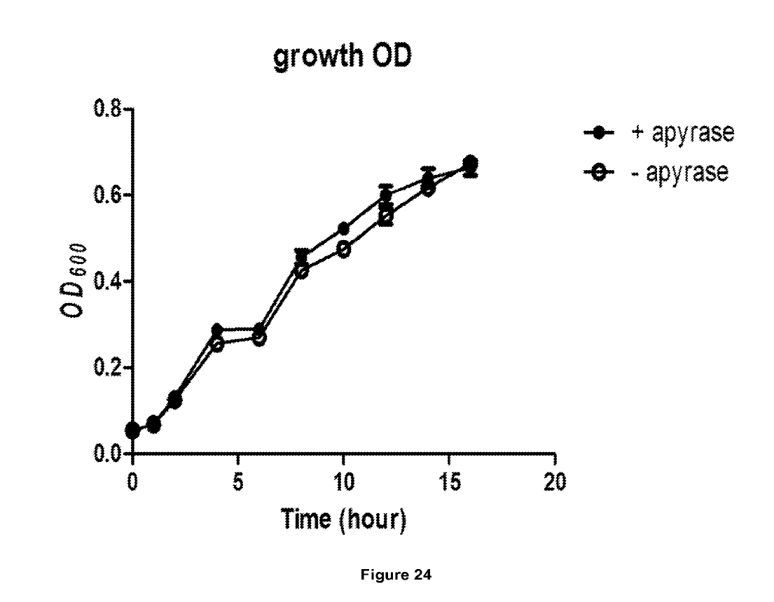

During the development of embodiments of the technology provided herein, experiments were conducted demonstrating that apyrase does not affect bacterial growth. As shown in FIG. 24, the growth of a bacterial culture (as monitored by measuring the optical density at 600 nm) was the same in the presence and absence of apyrase over the course of 15 to 20 hours.

### Example 25

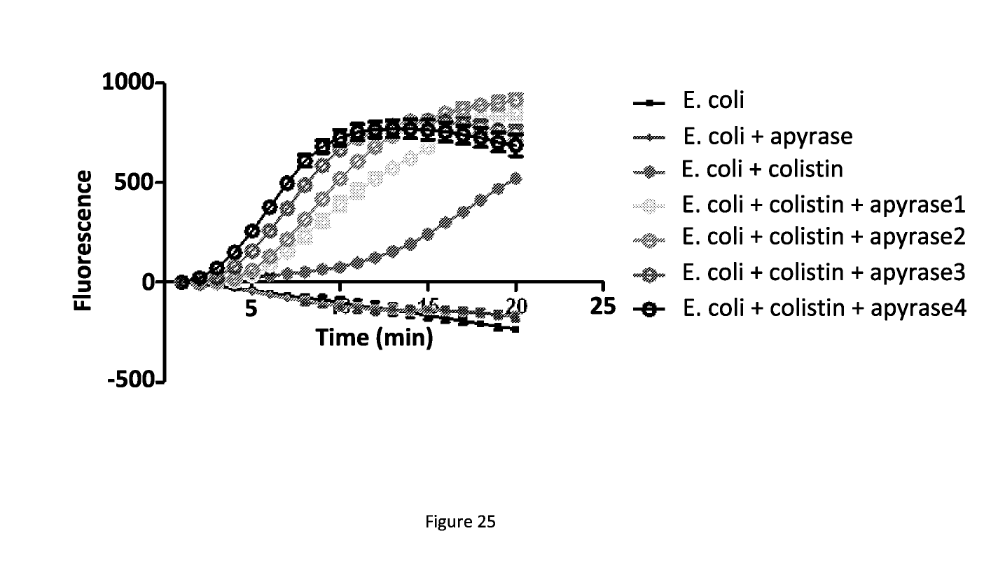

During the development of embodiments of the technology provided herein, experiments were conducted demonstrating that apyrase and an antimicrobial agent act to permeabilize the cell membranes of bacterial cells. Bacterial membrane permeabilization was conducted as described in Bourbon, et al., Analytical Biochemistry 381(2): 279-81 (2008), incorporated herein by reference. In short, the green fluorescent dye SYTOX is added to bacterial cells to measure membrane permeabilization. SYTOX fluorescence is low outside a cell and is substantially increased when it is in a cell interior. Thus, an increase in fluorescence represents an increase in permeability of the cell membrane. Using an E. coli culture of about 10⁷ CFU/ml, SYTOX was added to monitor permeability in the presence of apyrase at 2 U/ml and/or colistin at 0, 0.25, 0.5, 1, and 2 U/ml. As shown in FIG. 25, the increase of SYTOX fluorescence indicates increased permeabilization of the bacterial membrane in the presence of colistin and apyrase.

### Example 26

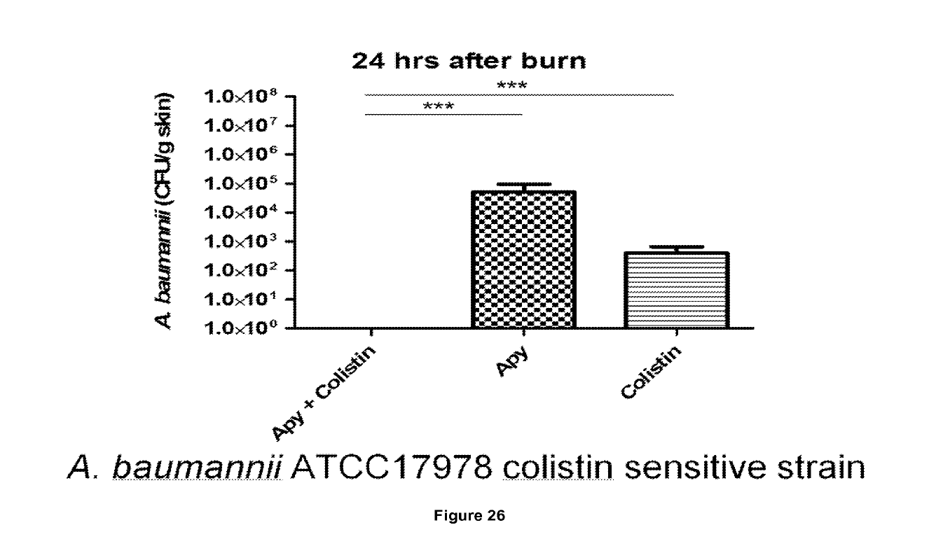

During the development of embodiments of the technology provided herein, experiments were conducted to establish the synergistic effects of apyrase and antimicrobial agents in vivo, e.g., in a mouse model for wound infection and healing. As shown in FIG. 26, the colistin-sensitive strain A. baumannii ATCC 17978 was not detected on skin 24 hours after a burn in the presence of apyrase and colistin. In the presence of apyrase or colistin alone, A. baumannii was detected at amounts from 10² to 10⁵ CFU/g of skin. In these experiments, colistin was used at 0.2 μg/ml.

### Example 27

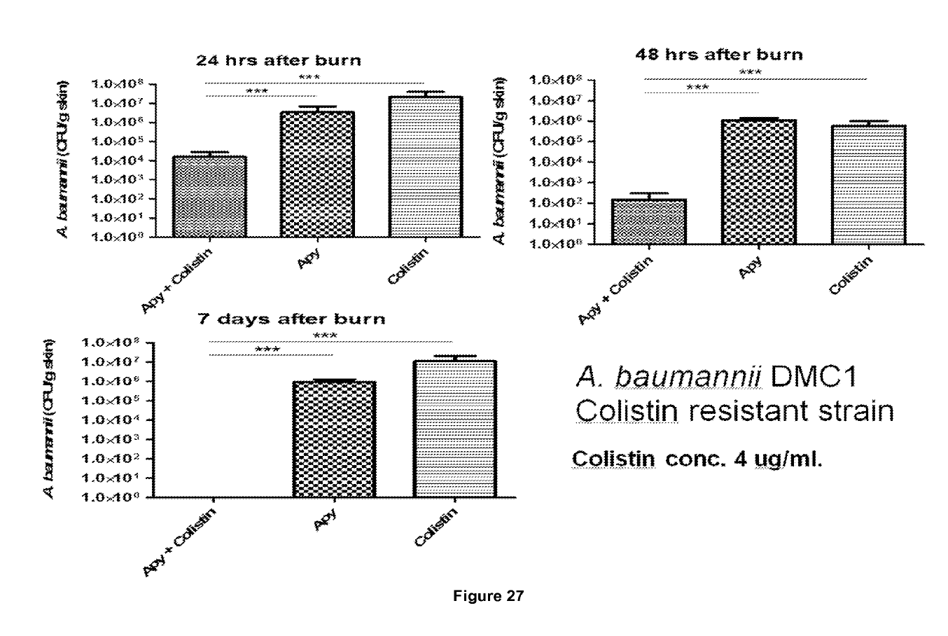

During the development of embodiments of the technology provided herein, experiments were conducted to establish the synergistic effects of apyrase and antimicrobial agents in vivo, e.g., in a mouse model for wound infection and healing. As shown in FIG. 27, the colistin-resistant strain A. baumannii ATCC DMC1 was decreasingly detected on skin 24 hours, 48 hours, and 7 days after a burn in the presence of apyrase and colistin. After 7 days, A. baumannii ATCC DMC1 was not detectable in the presence of apyrase and colistin. In the presence of apyrase or colistin alone, A. baumannii was detected at amounts from 10⁵ to 10⁸ CFU/g of skin throughout the test period over 7 days. In these experiments, colistin was used at 4 μg/ml.

### Example 28

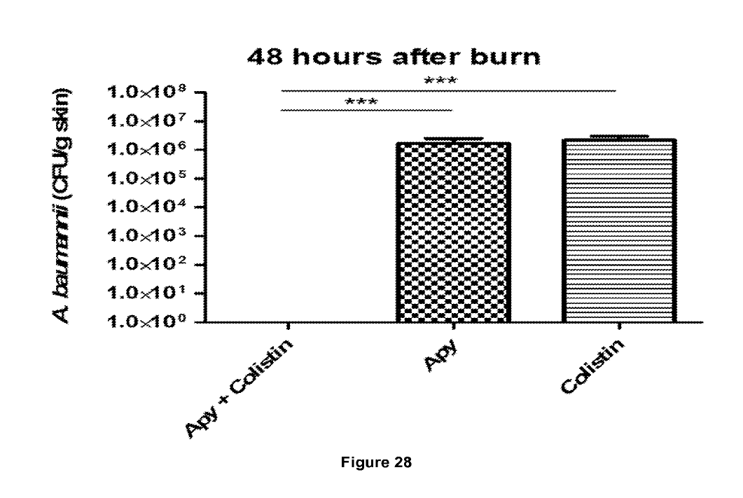

During the development of embodiments of the technology provided herein, experiments were conducted to establish the synergistic effects of apyrase and antimicrobial agents in vivo, e.g., in a mouse model for wound infection and healing. After a burn, A. baumannii ATCC DMC1 was inoculated on skin. After 24 hours, the wounds were treated with apyrase alone, colistin alone, or apyrase and colistin in combination. Skin samples were collected after another 24 hours and the CFU/g skin of A. baumannii ATCC DMC1 was determined. As shown in FIG. 28, A. baumannii ATCC DMC1 was not detectable in the presence of apyrase and colistin 48 hours after the burn. In the presence of apyrase or colistin alone, A. baumannii was detected at amounts of approximately 10⁸ CFU/g of skin.

### Example 29

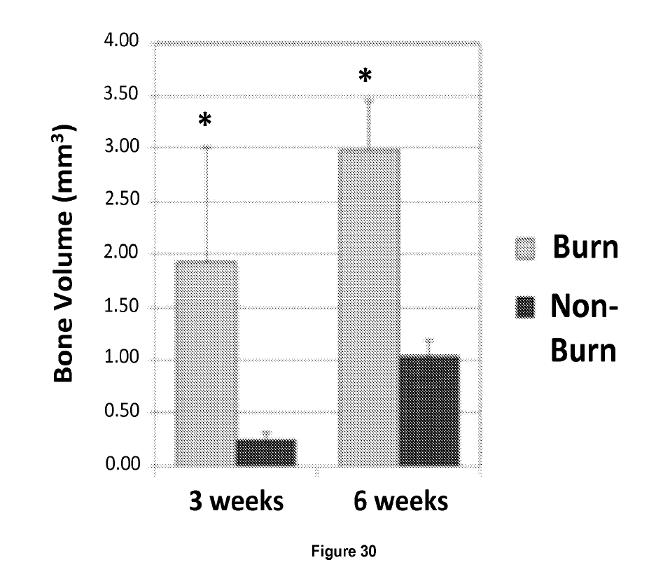

During the development of embodiments of the technology, the relationship of burn injury and heterotopic ossification were studied in vivo. In particular, a heterotopic ossification model using an achilles tenotomy was used to demonstrate that burn injury significantly drives heterotopic bone formation at the tenotomy site. The data demonstrate that after an Achilles tenotomy, mice with a burn injury develop significantly more bone and develop bone at an earlier time point than non-burn control mice (FIG. 30).

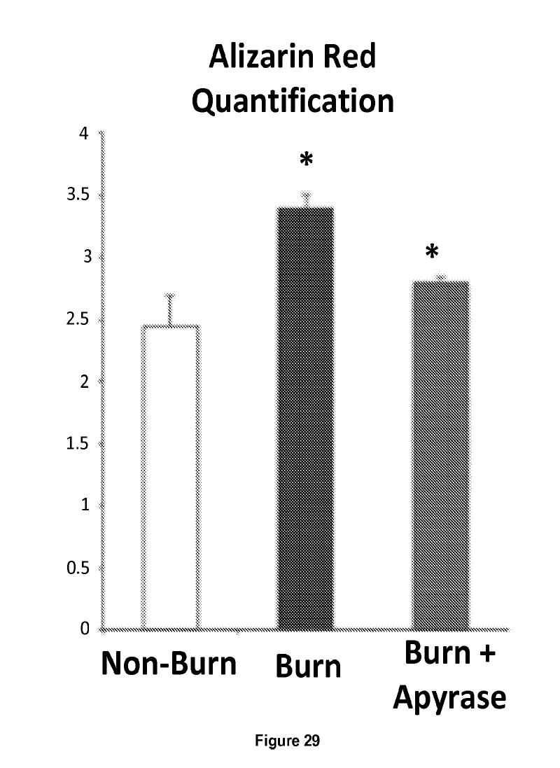

Multiple studies have demonstrated a link between acute inflammation and BMP-2 signaling, ultimately increasing bone formation. At the cellular level, research has shown that aberrant expression of BMPs in adipose-derived MSCs near the wound site stimulates osteogenic differentiation of multipotent cells. In support of this, data collected using the burn mouse model show an increase in osteogenic differentiation of MSCs after burn injury and a significant decrease of osteogenic differentiation of MSCs after burn injury and apyrase treatment (FIG. 29). Alizarin red is used in to determine, quantitatively by colorimetry, the presence of calcific deposition by cells of an osteogenic lineage. As such, it is a marker of matrix mineralization, a crucial step towards the formation of calcified extracellular matrix associated with true bone. Thus, alizarin red staining indicates differentiation of cells to bone lineages.

Consequently, it is contemplated that decreasing inflammation at the burn site with apyrase—thus decreasing ATP concentrations, which leads to decreased MSC BMP-2 signaling and decreased osteogenic differentiation—alleviates heterotopic ossification at wound sites. During the development of embodiments of the technology provided, data were collected demonstrating that by decreasing the inflammation at the burn site with apyrase, the osteogenic capacity and level of BMP-2 signaling of the MSCs is indeed decreased (FIG. 29).

Thus, in an effort to mitigate this osteogenic microenvironment created by acute inflammation, embodiments of the technology relate to treating (e.g., by topical application) wounds (e.g., a burn) to decrease inflammation by reducing ATP concentrations. The resulting decrease in inflammation at the wound or burn site with apyrase decreases the osteogenic capacity and level of BMP-2 signaling of the MSCs (FIG. 29). As such, it is contemplated that applying apyrase to the burn site at the time of the burn injury and Achilles tenotomy prevents heterotopic bone formation by decreasing inflammation at the burn injury site. Accordingly, apyrase finds use to inhibit mesenchymal stem cell osteogenesis through apyrase mediated suppression of burn inflammation.

### Example 30

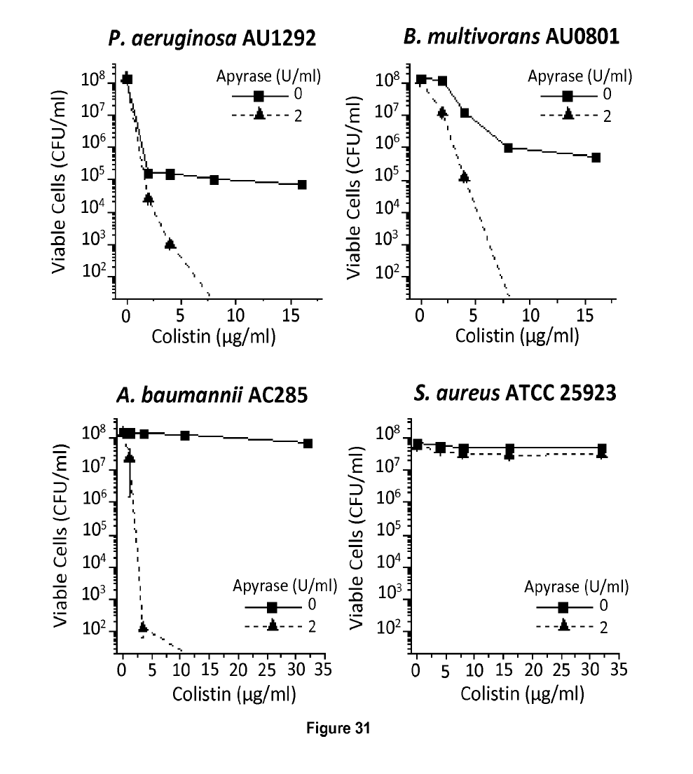

Experiments performed during the development of the technology provided herein demonstrated that apyrase treatment synergistically improves killing efficacy of colistin against Gram-negative ESKAPE pathogens. Data were collected from systematic tests to investigate bacterial killing with colistin and apyrase combinations against ESKAPE bacteria in vitro. Escherichia coli, Staphylococcus aureus, Klebsiella pneumonia, Acinetobacer baumannii, Pseudomonas aeruginosa, and Burkholderia species were tested for synergistic killing efficacy by colistin (colistin sulfate salt, C4461, Sigma-Aldrich, St. Louis, Mo.) and apyrase (M0393L, New England BioLabs Inc., Ipswich, Mass.). Dose-kill curves for 22 strains (colistin sensitive or resistant ˜10⁶ CFU/ml) were generated after exposure to colistin at different concentrations by itself or in combination with apyrase (2 U/ml) for 4 hours at 37° C. Data were obtained from three independent experiments. The results showed there were significant synergistic killing effects on all Gram-negative strains, regardless of colistin-sensitive or resistant isolates. The MIC of colistin and apyrase combinations was generally 2- to 8-fold lower in comparison to the MIC for colistin alone (FIG. 31 and Table 2). For Gram-positive strains, the MICs results obtained with colistin alone and with the combination of colistin and apyrase were very similar.

TABLE 2
synergistic killing of Pseudomonas, Burkholderia, Acinetobacter,
Escherichia, Klebsiella, and Staphylococcus.

| | Colistin | Colistin + Apyrase |
|---|---|---|
| Strains | MIC | MIC |
| P. aeruginosa AU1292 | >16 | 6 |
| P. aeruginosa AU7443 | >16 | 6 |
| P. aeruginosa ATCC 27853 | 1 | 0.25 |
| P. aeruginosa PAO300 | 2 | 0.75 |
| B. multivorans AU0801 | >16 | 8 |
| A. baumannii ATCC 19606 | 4 | 1.2 |
| A. baumannii DMC1 | >32 | 4 |
| A. baumannii VBA9 | >32 | 11 |
| A. baumannii AC285 | >32 | 12.5 |
| A. baumannii AC121 | >32 | 11 |
| K. pneumonia AD | 16 | 4 |
| E. coli K-12 | 2 | 0.125 |
| S. aureus ATCC 25923 | >32 | >32 |

In addition, experiments were performed to test the synergistic killing effects of colistin and apyrase in vivo. An established mouse wound model was used for this test.

Female pathogen-free C57BL/6 mice (Harlan, Indianapolis, Ind.), 12 weeks old, weighing ˜20-23 grams were used in all experiments. The skin over the lumbrosacral and back region was clipped using a 35-W model 5-55 E electrical clipper (Oyster-Golden A-S, Head no. 80, blade size 40) by using the method described by Ipaktchi et al. with slight modification. Two hundred microliters of a 0.9% saline suspension of A. baumannii DMC1 (1×10⁶ cells/ml), which is a highly colistin resistant clinical strain, was used for wound infection experiments. Viable bacterial numbers were determined 24 hours after burn. The Student t-test was used for statistical analysis.

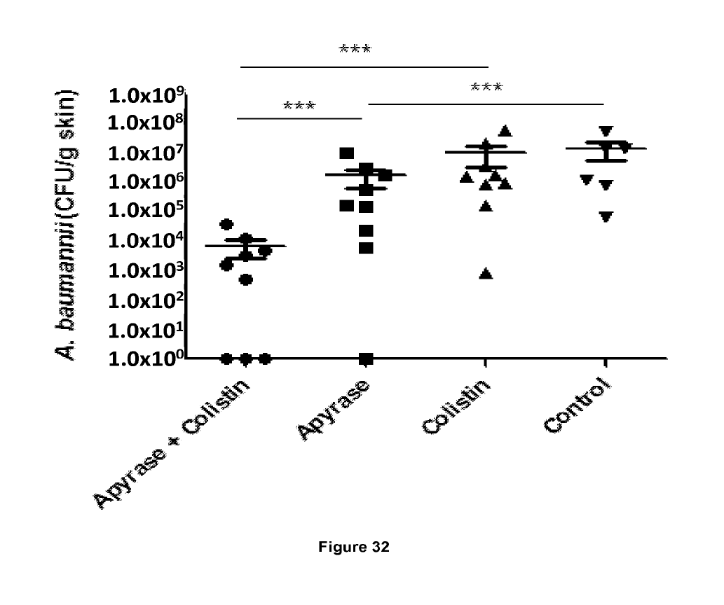

Experiments were carried out in four groups: (1) a control group (n=6), which contained neither apyrase nor colistin; (2) apyrase group (n=9), which contained apyrase (1 U/ml); (3) colistin group (n=9), which contained colistin (4 μg/ml); and (4) apyrase and colistin group (n=9), which was treated with the combination of apyrase (1 U/ml) and colistin (4 μg/ml). The treatment was applied immediately after inoculation. After 24 hours, the mice were given lethal IP injections of pentobarbital (150 mg/kg) and skin samples were collected using a scalpel and scissors. Data showed that colistin group and the control group had the highest viable bacteria number recovered from burn sites 24 hours after burn and those two groups have no significant difference. Apyrase group had about a 1-log reduction in viable bacteria number compared to control and colistin groups. The combination group had the lowest viable bacteria number in all four groups: a 2-log reduction in bacteria compared to the apyrase group and a 3-log reduction in comparison with the control and colistin groups (FIG. 32; *** indicates significant difference at P<0.01). This result demonstrated that apyrase alone has the ability to reduce bacterial infection when applied to wounds and exhibits a synergistic killing effect against bacteria that are resistant to colistin.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in pharmacology, biochemistry, medical science, or related fields are intended to be within the scope of the following claims.

1.-36. (canceled)

37. A composition comprising:
a) apyrase; and
b) an antimicrobial agent.

38. The composition of claim 37 wherein the antimicrobial agent is colistin or an antibacterial polymer.

39. The composition of claim 38 wherein the antibacterial polymer is polymer E2 or polymer E4.

40. The composition of claim 37 wherein the apyrase has a concentration of from approximately 1 to approximately 10 U/ml.

41. The composition of claim 37 wherein the antimicrobial agent belongs to a class selected from the group consisting of macrolide, penicillin, cephalosporin, carbepenem, monobactam, beta-lactam inhibitor, oxaline, aminoglycoside, chloramphenicol, sulfonamide, glycopeptides, quinolone, tetracycline, fusidic acid, trimethoprim, metronidazole, clindamycin, mupirocin, rifamycins, streptogramin, lipoprotein, polyene, azole, and echinocandin.

42. The composition of claim 37 wherein the antimicrobial agent is selected from the group consisting of erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, and nystatin.

43. A method for treating or preventing an infection or colonization, the method comprising;
1) identifying a subject in need of an antimicrobial treatment; and
2) administering to the subject;
a) an apyrase and
b) an antimicrobial agent.

44. The method of claim 43 wherein the administering comprises administering a composition comprising the apyrase and the antimicrobial agent.

45. The method of claim 43 wherein the administering comprises administering a first composition comprising the apyrase and administering a second composition comprising the antimicrobial agent.

46. The method of claim 45 wherein the first composition and the second composition are administered sequentially.

47. The method of claim 45 wherein the first composition and the second composition are administered simultaneously.

48. The method of claim 43 further comprising testing the subject for an infection and/or colonization.

49. The method of claim 43 wherein the administering occurs by a route selected from the group consisting of oral, intravenous, intraperitoneal, intramuscular, transdermal, subcutaneous, topical, sublingual, and rectal.

50. The method of claim 43 wherein the administering comprises contacting a subject with a material selected from the group consisting of a dressing, a gel, an ointment, a bandage, a solution, a cream, a salve, and a spray, wherein the composition is a component of the material.

51. The method of claim 43 wherein the subject has a wound and/or a burn.

52. A method for treating a subject in need of a treatment for heterotopic ossification, wherein the method comprises:
1) identifying a subject in need of a treatment for heterotopic ossification; and
2) administering to the subject an agent that hydrolyzes a compound selected from the group consisting of ATP, dATP, an analog of ATP, and a derivative of ATP.

53. The method of claim 52 wherein the agent is an apyrase.

54. The method of claim 52 wherein the subject has experienced a trauma, burn, upper extremity injury, surgery, amputation, a central nervous system injury, a vasculopathy, an arthroplasty, or an end-stage cardiac valve disease.

55. A method of treating a wound or a burn, the method comprising:
1) selecting a subject who is in need of an antibacterial and an anti-osteogenic treatment; and
2) administering to the subject a composition comprising apyrase.
